(19) United States
(12) Patent Application Publication
(10) Pub. No.: US 2024/0008811 A1
(43) Pub. Date: Jan. 11, 2024

(54) USING ARTIFICIAL INTELLIGENCE TO DETECT AND MONITOR GLAUCOMA

(71) Applicant: ArcScan, Inc., Golden, CO (US)

(72) Inventors: Matthew G. Sassu, Highlands Ranch, CO (US); Johan Erik Giphart, Superior, CO (US)

(21) Appl. No.: 18/219,847

(22) Filed: Jul. 10, 2023

Related U.S. Application Data
(60) Provisional application No. 63/359,628, filed on Jul. 8, 2022.
(60) Provisional application No. 63/418,890, filed on Oct. 24, 2022.
(60) Provisional application No. 63/417,590, filed on Oct. 19, 2022.

(51) Int. Cl. A61B 5/00; A61B 3/16; A61B 3/14; A61B 3/117; A61B 3/10; A61B 3/12; G16H 50/20; G06T 7/00

(52) U.S. Cl. CPC A61B 5/4842; A61B 3/16; A61B 3/14; A61B 3/117; A61B 3/1005; A61B 3/1216; G16H 50/20; G06T 7/0014; G06T 2207/30041; G06T 2207/20081

(57) ABSTRACT

Methods, systems, and devices include locating one or more target structures comprised in an eye of a patient based on processing image data of the eye of the patient, determining one or more measurements associated with an anterior portion of the eye based on the location data, and determining a presence, an absence, a progression, or a stage of a disease of the eye based on the one or more measurements. Locating the one or more target structures may be based on an output provided by a machine learning model.

### CROSS-REFERENCE TO RELATED APPLICATIONS

The present application claims the benefit of U.S. Provisional Application Ser. No. 63/359,628 filed Jul. 8, 2022, U.S. Provisional Application Ser. No. 63/417,590 filed Oct. 19, 2022 and U.S. Provisional Application Ser. No. 63/418,890 filed Oct. 24, 2022. The entire disclosures of the applications listed are hereby incorporated by reference, in their entirety, for all that the disclosures teach and for all purposes.

### FIELD OF TECHNOLOGY

The following relates to medical imaging of the eye and, in particular, medical imaging in association with detecting and monitoring a disease of the eye.

### BACKGROUND

Some systems may support medical imaging techniques of the eye for examination or therapeutic purposes. Techniques supportive of detecting or monitoring disease of the eye based on imaging data are desired.

### SUMMARY

The described techniques relate to improved methods, systems, devices, and apparatuses that support medical imaging of an anterior segment of the eye in association determining a presence, an absence, a progression, or a stage of a disease of the eye.

In some aspects, the techniques described herein relate to a method including: locating one or more target structures included in an eye of a patient based on processing image data of the eye of the patient, wherein processing the image data includes: providing at least a portion of the image data to one or more machine learning models; and receiving an output from the one or more machine learning models in response to the one or more machine learning models processing at least the portion of the image data, wherein the output includes location data of the one or more target structures; determining one or more measurements associated with an anterior portion of the eye, based on the location data and one or more characteristics associated with the one or more target structures; and determining a presence, an absence, a progression, or a stage of a disease of the eye based on the one or more measurements.

In some aspects, the techniques described herein relate to a method, wherein determining the presence, the absence, the progression, or the stage is based on a correlation between the one or more measurements and the disease.

In some aspects, the techniques described herein relate to a method, further including: providing the one or more measurements to the one or more machine learning models; and receiving a second output in response to the one or more machine learning models processing the one or more measurements, wherein: the second output includes a probability of the disease of the eye; and determining the presence, the absence, the progression, or the stage is based on the probability.

In some aspects, the techniques described herein relate to a method, wherein: the output from the one or more machine learning models includes one or more predicted masks; and determining the location data, the one or more measurements, or both is based at least in part on the one or more predicted masks.

In some aspects, the techniques described herein relate to a method, wherein the one or more measurements include at least one of: a measurement with respect to at least one axis of a set of axes associated with the eye; an angle between two or more axes of the set of axes; and a second measurement associated with an implant included in the eye.

In some aspects, the techniques described herein relate to a method, wherein the one or more target structures include at least one of: tissue included in the eye; surgically modified tissue included in the eye; pharmacologically modified tissue included in the eye; and an implant included in the eye.

In some aspects, the techniques described herein relate to a method, further including: determining a change in intraocular pressure in the eye based on the one or more measurements, wherein determining the presence, the absence, the progression, or the stage of the disease is based on the intraocular pressure.

In some aspects, the techniques described herein relate to a method, wherein: the one or more measurements are associated with a first region posterior to an iris of the eye, a second region anterior to the iris, or both.

In some aspects, the techniques described herein relate to a method, wherein: the image data includes one or more images generated based on one or more imaging signals, the one or more imaging signals including ultrasound pulses; and the image data includes a B-scan of the eye of the patient.

In some aspects, the techniques described herein relate to a method, wherein: the image data includes one or more images generated based on one or more imaging signals, the one or more imaging signals including infrared laser light; and the image data includes a B-scan of the eye of the patient.

In some aspects, the techniques described herein relate to a method, wherein the one or more measurements include at least one of: anterior chamber depth; iris thickness; iris-to-lens contact distance; iris zonule distance; trabecular ciliary process distance; and trabecular iris space area; and a measurement associated with an implant included in the eye.

In some aspects, the techniques described herein relate to a method, further including training the one or more machine learning models based on a training data set, the training data set including at least one of: reference image data associated with at least one eye of one or more reference patients; label data associated with the one or more target structures; one or more reference masks for classifying pixels included in the reference image data in association with locating the one or more target structures; and image classification data corresponding to at least one image of a set of reference images, wherein the reference image data, the label data, the one or more reference masks, and the image classification data are associated with a pre-operative state, an intraoperative state, a post-operative state, a disease state, or a combination thereof.

In some aspects, the techniques described herein relate to a method, wherein: the image data includes a set of pixels; and processing at least the portion of the image data by the one or more machine learning models includes: generating encoded image data in response to processing at least the portion of the image data using a set of encoder filters; and generating a mask image in response to processing at least the portion of the encoded image data using a set of decoder filters, wherein the mask image includes an indication of one or more pixels, included among the set of pixels included in the image data, that are associated with the one or more target structures.

In some aspects, the techniques described herein relate to an apparatus including: a processor; and memory in electronic communication with the processor, wherein instructions stored in the memory are executable by the processor to: locate one or more target structures included in an eye of a patient based on processing image data of the eye of the patient, wherein processing the image data includes: providing at least a portion of the image data to one or more machine learning models; and receiving an output from the one or more machine learning models in response to the one or more machine learning models processing at least the portion of the image data, wherein the output includes location data of the one or more target structures; determine one or more measurements associated with an anterior portion of the eye, based on the location data and one or more characteristics associated with the one or more target structures; and determine a presence, an absence, a progression, or a stage of a disease of the eye based on the one or more measurements.

In some aspects, the techniques described herein relate to an apparatus, wherein determining the presence, the absence, the progression, or the stage is based on a correlation between the one or more measurements and the disease.

In some aspects, the techniques described herein relate to an apparatus, wherein the instructions are further executable by the processor to: provide the one or more measurements to the one or more machine learning models; and receive a second output in response to the one or more machine learning models processing the one or more measurements, wherein: the second output includes a probability of the disease of the eye; and determining the presence, the absence, the progression, or the stage is based on the probability

In some aspects, the techniques described herein relate to an apparatus, wherein: the output from the one or more machine learning models includes one or more predicted masks; and determining the location data, the one or more measurements, or both is based at least in part on the one or more predicted masks.

In some aspects, the techniques described herein relate to an apparatus, wherein the one or more measurements include at least one of: a measurement with respect to at least one axis of a set of axes associated with the eye; an angle between two or more axes of the set of axes; and a second measurement associated with an implant included in the eye.

In some aspects, the techniques described herein relate to an apparatus, wherein the one or more target structures include at least one of: tissue included in the eye; surgically modified tissue included in the eye; pharmacologically modified tissue included in the eye; and an implant included in the eye.

In some aspects, the techniques described herein relate to a non-transitory computer readable medium including instructions, which when executed by a processor: locates one or more target structures included in an eye of a patient based on processing image data of the eye of the patient, wherein processing the image data includes: providing at least a portion of the image data to one or more machine learning models; and receiving an output from the one or more machine learning models in response to the one or more machine learning models processing at least the portion of the image data, wherein the output includes location data of the one or more target structures; determines one or more measurements associated with an anterior portion of the eye, based on the location data and one or more characteristics associated with the one or more target structures; and determines a presence, an absence, a progression, or a stage of a disease of the eye based on the one or more measurements.

### BRIEF DESCRIPTION OF THE DRAWINGS

FIG. 1 illustrates the anatomy of the eye in a region near a scleral spur.

FIG. 2 illustrates an angle opening distance (AOD) measured in accordance with aspects of the present disclosure.

FIG. 3 illustrates example measurements in accordance with aspects of the present disclosure.

FIG. 4 illustrates an example architecture of a neural network that supports generating a mask image in accordance with aspects of the present disclosure.

FIG. 5 illustrates an example image generated based on imaging signals associated with an imaging device in accordance with aspects of the present disclosure.

FIG. 6 illustrates an example mask image generated using a neural network in accordance with aspects of the present disclosure.

FIG. 7 illustrates an example image generated based on imaging signals associated with an imaging device in accordance with aspects of the present disclosure.

FIG. 8 illustrates an example mask image generated using a neural network in accordance with aspects of the present disclosure.

FIG. 9 illustrates example anatomy detected using techniques supported by aspects of the present disclosure.

FIG. 10 illustrates an example of an interface line between the scleral wall and a ciliary muscle.

FIG. 11 illustrates an example of a system supportive of the techniques described herein in accordance with aspects of the present disclosure.

FIG. 12 illustrates an example apparatus in accordance with aspects of the present disclosure.

FIG. 13 and FIG. 14 illustrate example process flows supportive of aspects of the present disclosure.

### DETAILED DESCRIPTION

Aspects of the present disclosure may take form in various components and arrangements of components, and in various steps and arrangements of steps. The drawings illustrate aspects of one or more example embodiments supported by aspects of the present disclosure and are not to be construed as limiting the invention. In the drawings, like reference numerals may refer to like or analogous components throughout the several views.

Aspects of the present disclosure relate to systems and techniques which, using imaging data of the anterior segment of the eye, coupled with artificial intelligence algorithms for automatically locating anatomy in the eye, support identifying landmarks (e.g., scleral spur). The systems and techniques support, using the landmarks as a fiduciary, automatically making measurements in front of and behind the iris. The systems and techniques support detecting and monitoring a disease (e.g., glaucoma, etc.) of the eye based on the measurements.

Glaucoma is a group of diseases that cause optical nerve damage and can eventually lead to blindness. In some cases, the early stages of glaucoma may not result in any symptoms and, as a result patients may be unaware of the disease due to the lack of symptoms. The leading risk factor for glaucoma is intraocular pressure (IOP). Intraocular pressure is the pressure in the eye created by the balance between continual renewal of fluids within the eye and drainage of fluids from the eye. For example, for a stable state with respect to intraocular pressure, fluid generated equals fluid drained.

In some cases, intraocular pressure may be affected by changes in fluid generation or drainage structures (e.g., when Schlemm's canal and trabecular mesh through which the fluid normally drains becomes progressively blocked). When diagnosed in the early stages, progression of glaucoma can be halted by medication or surgical treatments. Specific treatment may depend on the stage and type of glaucoma. Example types of glaucoma include acute (angle closure) glaucoma, chronic (open-angle) glaucoma, normal tension glaucoma, and secondary glaucoma.

Some tests for measuring the pressure in the eye include tonometry tests. However, tonometry fails to provide information about factors causing abnormal pressure. Imaging the anterior segment of the eye may help identify the type and causes of glaucoma (e.g., whether the glaucoma is open-angle or angle closure glaucoma). Furthermore, through imaging, subtle anatomical changes can be visualized, measured, and tracked over time possibly even before other measurable changes (e.g., intraocular pressure, nerve damage) occur.

Gonioscopy is a qualitative test where a lens with special prisms is placed on the eye to visually inspect the drainage angle of the eye, determine whether the drainage angle is open or closed, and determine to what degree if the drainage angle is closed. The examination associated with gonioscopy can be somewhat uncomfortable for a patient, may require numbing, and requires skill and subjective judgment on the part of medical personnel.

To improve upon the subjectivity of gonioscopy, some techniques for diagnosing the onset and progression of glaucoma include imaging the anterior segment of the eye using optical and/or ultrasound instruments. As will be described herein, using optical instruments and/or ultrasound technologies, systems and techniques are described which enable medical personnel to make one or more quantitative measurements (e.g., iridocorneal angle, anterior chamber depth, iris/lens contact distance, iris/zonule distance, and trabecular ciliary process distance) and/or autonomously determine the measurements and provide the same to the medical personnel.

One imaging technology is optical Coherence Tomography (OCT) which is a light-based imaging technology that can image most of the cornea. OCT cannot see clearly behind the scleral wall or at all behind the iris and is therefore of reduced use in screening for the early onset of glaucoma. OCT does well for imaging the central retina although only to the lateral extent allowed by a dilated pupil.

Ultrasound Bio Microscopy (UBM) is currently the most common means of ultrasound imaging of the anterior segment of the eye. A UBM can capture anterior segment images using a transducer capable of emitting very high frequency acoustic pulses ranging from about 20 to about 80 MHz. UBM may be implemented with a handheld device. In some cases, the handheld device is used with an open scleral shell filled with saline, in which the open scleral shell is placed on an anesthetized eye and the UBM probe is held in the saline. AI ternately, in some UBM approaches, a Prager cup can be used. The procedure using a UBM may be uncomfortable for the patient, and the pressure of the UBM on the cornea can distort the cornea and eyeball.

The UBM method can provide qualitative ultrasound images of the anterior segment of the eye but cannot make accurate, precision, comprehensive, and measurable images of the cornea, lens or other components of the eye required for glaucoma screening, keratoconus evaluation or lens sizing for two reasons. First, a UBM device is a hand-held device and relies on the steadiness of the operator's hand to maintain a fixed position relative to the eye being scanned for several seconds. Furthermore, placing the ultrasound beam over an exact location may be difficult, and especially repeatably so in the case of repeat examinations (e.g., for repeat examinations at annual intervals). Second, to make contact with the cornea of the patient to obtain an acoustic coupling satisfactory for UBM, the UBM device is pressed firmly onto the eye of the patient. The resultant pressure gives rise to some distortion of the cornea and the eyeball.

Ultrasonic imaging can be used to provide accurate images in the corner of the eye in the region around the junction of the cornea, the sclera, and the iris (e.g., in the region of the suprachoroidal space to the scleral spur), which is well off-axis and essentially inaccessible to optical imaging. Other procedures such as implantation of stents in or near the suprachoroid may provide part or all of a treatment for glaucoma.

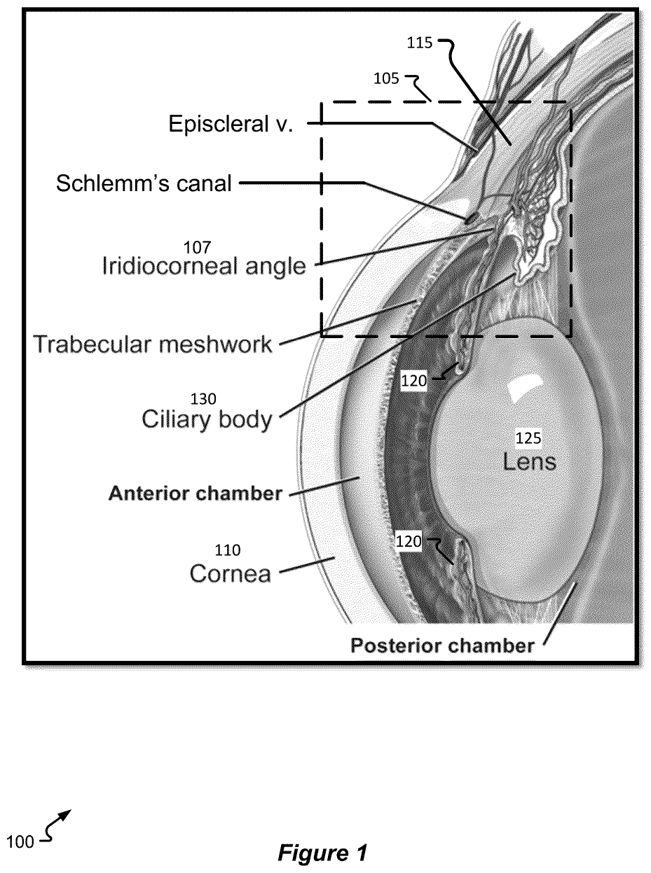

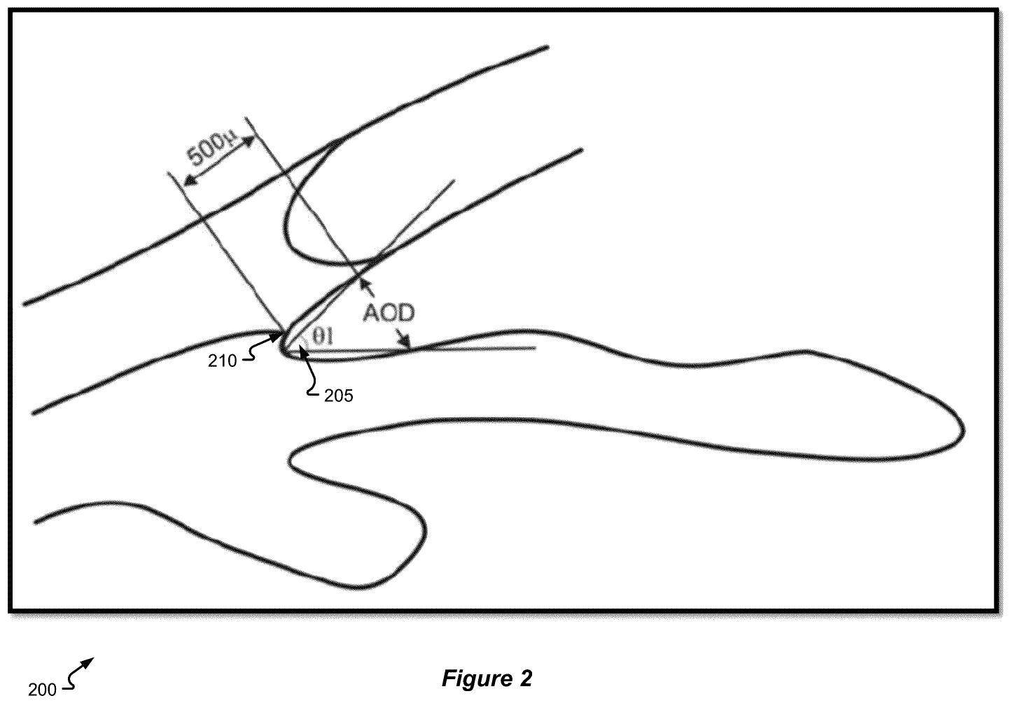

The region of the eye where the cornea, iris, sclera and ciliary muscle are all in close proximity is illustrated in FIGS. 1 and 2. FIGS. 1 and 2 illustrate the iridocorneal angle, scleral spur, trabecular mesh and ciliary process, for example.

Precision ultrasound imaging with an arc scanner (for example as described in U.S. Pat. No. 8,317,702) in the frequency range of about 5 MHz to about 80 MHz can be applied to make more accurate, precise and repeatable measurements of structures of the eye, such as, for example, the cornea and lens capsule, ciliary muscle and the like. Such measurements provide an ophthalmic surgeon with valuable information that can be used to guide various surgical procedures for correcting refractive errors in LASIK and lens replacement procedures. They also provide diagnostic information after surgery to assess the geometrical location of corneal features (e.g., LASIK scar) and lens features (e.g., lens connection to the ciliary muscle, lens position and lens orientation). The arc scanning ultrasound system is capable of accurately moving an ultrasound transducer with respect to a known reference point on the head of a patient.

Precision ultrasonic imaging may involve a liquid medium to be interposed between the object (e.g., eye of the patient) being imaged and the transducer, in which the object, the transducer, and the path between the object and the transducer be at all times be immersed in the liquid medium. An eyepiece serves to complete a continuous acoustic path for ultrasonic scanning, that path extending from the transducer to the surface of the eye of the patient. The eyepiece also separates the water in which the eye of the patient is immersed from the water in the chamber in which the ultrasound transducer and guide track assembly are contained. The eyepiece provides a steady rest for the patient and helps the patient to remain steady during a scan. The eyepiece should be free from frequent leakage problems, should be comfortable to the patient and its manufacturing cost should be low since it should be replaced for every new patient.

According to example aspects of the present disclosure, techniques described herein may utilize a precision ultrasound scanning device to detect the onset and progression of glaucoma by imaging structural changes in the anterior segment before any retinal damage occurs. The techniques described herein may utilize the imaged structural changes to identify the onset and/or progression of the disease, which may enable successful treatment (e.g., with drugs and/or stent implants).

The systems and techniques described herein incorporate a precision ultrasound scanning device, coupled with artificial intelligence algorithms, capable of automatically locating the anatomical regions and landmarks (e.g., tissue, surgically modified tissue, pharmacologically modified tissue, an implant, etc.) in the eye of a patient by imaging through the scleral wall and through the iris. In some aspects, using location information of the anatomical regions and landmarks, the systems and techniques may autonomously provide measurements having increased accuracy compared to other techniques, and the systems and techniques support repeatably providing such measurements. Using the measurements, the systems and techniques described herein may provide improved detection of changes in the eye that can precede elevation of intraocular pressure that characterizes the onset of glaucoma.

The various embodiments and configurations of the present disclosure are directed generally to medical imaging of the eye, in particular, medical imaging of an anterior segment of the eye in association with detecting and monitoring a disease of the eye. For example, the systems and techniques described herein relate generally to ultrasonic imaging of a target anatomy (e.g., cornea, sclera, iris, lens, ciliary process, scleral spur, etc.) in the anterior segment of an eye and, in particular, support a method for automatically locating the target anatomy using an artificial intelligence algorithm. Using the target anatomy (e.g., scleral spur, etc.) as a fiduciary, the systems and techniques support automatically making measurements in front of and behind the iris. The systems and techniques support detecting and monitoring a disease (e.g., glaucoma, etc.) of the eye based on the measurements. The terms “target anatomy” and “target structure” may be used interchangeably herein.

Arc scanning machines have demonstrated that they can repeatedly produce an image of eye features as small as about 5 microns in the depth direction (z-direction) and about 50 microns in either lateral direction (x- and y-directions) For example, scans of a cornea using an arc scanning machine can image the epithelial layer, Bowman's layer, and LASIK flap scars, all in a cornea that is about 500 microns thick. Thus it is important to be able to account for any unintended motions of the patient's head or eye during a scan, especially if multiple scans are made and later spliced together to form a composite image. An example allowing for tracking of unintended eye motions during scanning is disclosed in U.S. Pat. No. 9,597,059 entitled, “Tracking Unintended Eye Movements in an Ultrasonic Scan of the Eye.”

Aspects of the present disclosure include generating or acquiring imaging data of the anterior segment of the eye using an imaging device. In an example, the imaging device may be a focused ultrasonic transducer. A focused ultrasonic transducer has an aperture which is slightly concave with radius of curvature that focuses the acoustic pulses at a desired location. In an example case, a transducer with a diameter of 5 mm, a focal length of 15 mm, and a center frequency of 38 MHz, the depth of focus is about 1,560 microns.

In some aspects, an imaging device implemented in accordance with aspects of the present disclosure may have a transducer with a concave aperture. In some cases, image quality of acquired images may be relatively highest when the focal plane of the transducer is as close to the feature of interest as possible. Obtaining a strong, sharp image of an eye feature of interest involves fulfilling at least 2 conditions: (1) the focal plane is located near the feature of interest (e.g., within a threshold distance) and (2) the transducer pulse engages the surface of interest substantially normal to (e.g., in a direction substantially perpendicular to) the surface. In an example, condition (2) can be fulfilled by transmitting an imaging signal (e.g., ultrasound signal, etc.) such that the pulse wave train of the imaging signal passes through both the center of curvature of the transducer arcuate track guide and the center of curvature of the eye component surface.

One of the applications of a precision ultrasound scanning device or instrument is to image the region of the eye where the cornea, iris, sclera and ciliary muscle are all in close proximity (see FIG. 1). As supported by the systems and techniques described herein, using a knowledge of the structure of the eye in the region, along with analysis by artificial intelligence algorithms, some measurements can be made immediately, and the scleral spur located with only minimal additional processing. Once the position of the scleral spur and surrounding anatomical regions are determined, the systems and techniques support making additional measurements, using the scleral spur (or other anatomy described herein) as a fiduciary, that characterize the normal and abnormal shapes of elements within the anterior segment of the eye.

The systems and techniques support monitoring the measurement values over time. For example, over time, changes in the measurement values can indicate a change, or be a precursor for a change, of intraocular pressure (IOP). The systems and techniques described herein may support determining an onset, a presence, an absence, or a progression of a disease (e.g., glaucoma, etc.) of the eye based on the changes in measurement values or trends associated with the measurement values. Some examples of the measurements include corneal thickness, angle kappa, anterior and/or posterior radii of the cornea, anterior radii, posterior radii, and thickness of the natural lens, and posterior cornea to anterior lens distance along the visual axis, but are not limited thereto. It is to be understood that aspects described herein of measuring a radius support other related measurements (e.g., diameter).

Some non-limiting examples of anatomical changes utilized by the systems and techniques described herein in association with determining intraocular pressure include (but are not limited to):
Increases in corneal thickness.
Increases in the angle kappa.
The cornea bulges out, changing the anterior and posterior radii.
The natural lens compresses, changing anterior and posterior radii, and lens thickness.
Increases in the posterior cornea to anterior lens distance along visual axis.

Additionally, different Glaucoma treatments and surgeries can affect the anatomy in the eye. Aspects of the present disclosure support AI techniques of detecting, monitoring, and tracking changes in the anatomy. Non-limiting examples of the changes trackable by the systems and techniques described herein include:
Schlemm's Canal and the trabecular meshwork/collector channels.
Laser ablated tissue, for example the ciliary body.
Blebs
Shunts
The Suprachoroidal Space.

The techniques described herein support the ability to measure the described anatomy and any changes quickly, precisely, and reproducibly, as measuring the anatomy and any changes can be critical for: timely identification of a change in intraocular pressure, providing treatment to the condition over time, and preventing Glaucoma before it advances to irreversible nerve damage and blindness.

The AI based anatomy detection techniques from image data as described herein provide several advantages over other techniques. For example, in some other methods, the initial detection of anatomy in the B-Scan may be more computationally expensive compared to the techniques described herein. In an example, such methods may involve many checks to be sure the correct anatomy is being measured, resulting in increased processing overhead (e.g., increased processing time, increased processing complexity, increased processing costs due to hardware involved, etc.) compared to the techniques described herein.

By comparison, using neural networks, the systems and techniques support increased speed associated with processing an image and identifying anatomy. In an example, using neural networks, the systems and techniques may support processing an image and identifying anatomy in under a second. For example, some other techniques (e.g., as described in U.S. Pat. No. 11,357,479) for anatomy detection include processing image data (e.g., a B-scan) by binarizing the image data, and the techniques described herein may provide reduced processing overhead, increased speed, and increased accuracy in comparison. In some aspects, other techniques do not incorporate trained machine learning models for processing the image data and detecting anatomy from the image data.

In another example, using AI models, the systems and techniques may provide increased reliability associated with identifying anatomy and will not be inhibited by artefacts and/or anatomical anomalies present in image data. For example, B-Scans may be susceptible to multiple artifacts which may hinder anatomy identification from the B-scans.

In some other aspects, poor image quality may interfere with detection of anatomy, and AI based anatomy detection and identification may support automatic measurement of the anatomy that might otherwise be prevented if AI is not utilized. The techniques described herein provide a robustness supportive of immediate capture of measurements after the AI analysis (e.g., AI based anatomy detection and identification), without additional image processing steps or additional steps for verifying the region. Such speed and robustness improvements decrease the amount of time that operator spends analyzing data, which enables the operator to focus on treatment and increases patient throughput.

In some example implementations, the methods and techniques disclosed herein may include performing the following operations (in some cases, autonomously or semi-autonomously):
1. Acquire Image Data of the eye.
2. Using AI, locate target anatomy present in the image. At a minimum, for the steps included, the target anatomy may include the cornea, iris, natural lens, and scleral wall. It is to be understood that the target anatomy is not limited thereto, and the systems and techniques may support locating any appropriate anatomy in association with determining the measurements described herein.
3. Using the AI detected location of the iris, measure the iris thickness (ID).
4. Using the AI determined positions of the natural lens and cornea, measure the anterior chamber depth (ACD).
5. Using the AI determined positions of the natural lens and iris, determine the iris/lens contact distance (ILCD).
6. Using the AI determined locations of the iris and scleral wall, locate the iridocorneal angle.
7. Using the AI determined locations of the iris and scleral wall, locate the scleral spur along the inner scleral wall, near the angle.
8. Calculate the angle opening distance (AOD), located 500 microns from the close of the angle, or the scleral spur, depending on the analysis being performed.
9. Using the AI determined position of the scleral wall, locate root of the ciliary sulcus.
10. Using the scleral spur, iridocorneal angle, or other AI located anatomy as a fiduciary, make measurements including, but not limited to, the following:
a. The iris zonule distance (IZD). Note that the imaging method must be capable of imaging through the iris.
b. The trabecular ciliary process distance (TCPD). Note that the imaging method must be capable of imaging through the iris.
c. The trabecular iris area (TIA).
d. The iris-lens angle (ILA).

In accordance with aspects of the present disclosure, it is to be understood that 1 through 10 may be performed in a different order than the order illustrated, or may be performed in different orders or at different times. Certain operations (e.g., one or more of 1 through 10) may also be omitted, or one or more operations may be repeated, or other operations may be added to the operations. In some cases, 1 through 10 may be implemented as principal steps associated with anatomy detection and identification, measurements based on the anatomy, and detection/monitoring of a disease based on the measurements.

The following definitions are used herein:

An acoustically reflective surface or interface is a surface or interface that has sufficient acoustic impedance difference across the interface to cause a measurable reflected acoustic signal. A specular surface is typically a very strong acoustically reflective surface.

The angle kappa is the positive angle formed between the optical and visual axes.

The angle, or the iridocorneal angle, as referred to herein is the angle between the iris, which makes up the colored part of the eye, and the cornea, which is the clear-window front part of the eye. The angle is short for the iridocorneal angle. When the angle is open, most, if not all, of the eye's drainage system can be seen by using a special mirrored lens. When the angle is narrow, only portions of the drainage angle are visible, and in acute angle-closure glaucoma, none of it is visible. The angle is the location where the fluid that is produced inside the eye, the aqueous humor, drains out of the eye into the body's circulatory system. The function of the aqueous humor is to provide nutrition to the eye and to maintain the eye in a pressurized state. Aqueous humor should not be confused with tears, since aqueous humor is inside the eye.

The angle of opening, called the trabecular-iris angle (TIA), is defined as an angle measured with the apex in the iris recess and the arms of the angle passing through a point on the trabecular meshwork located 500 m from the scleral spur and the point on the iris perpendicularly. The TIA is a specific way to measure the angle or iridocorneal angle.

Anterior means situated at the front part of a structure; anterior is the opposite of posterior. The Anterior Chamber is the aqueous humor-filled space inside the eye between the iris and the cornea's endothelium (inner) surface. The Anterior Segment is the forward third of the eye, containing the Anterior Chamber and natural lens.

ArtificialIntelligence (“AI”) leverages computers and machines to provide problem-solving and decision-making capabilities. These systems are able to perform a variety of tasks (e.g., visual perception, object detection, speech recognition, decision-making, translation between languages, etc.). In medical diagnostics, AI can be used to aid in the diagnosis of patients with specific diseases. In medical imaging such as ultrasound and OCT, AI may be used to analyze images and identify features and artifacts. When researchers, doctors and scientists input data into computers, the newly built algorithms can review, interpret and even suggest solutions to complex medical problems.

An A-scan is a representation of a rectified, filtered reflected acoustic signal as a function of time, received by an ultrasonic transducer from acoustic pulses originally emitted by the ultrasonic transducer from a known fixed position relative to an eye component.

The anterior segment comprises the region of the eye from the cornea to the back of the lens.

Automatic refers to any process or operation done without material human input when the process or operation is performed. A process or operation can be automatic, even though performance of the process or operation uses material or immaterial human input, if the input is received before performance of the process or operation. Human input is deemed to be material if such input influences how the process or operation will be performed.

A bleb is a fluid filled blister that develops on the surface of eye. The fluid is mostly serous in nature. It can be on the white of an eye, conjunctiva or on the corneal portion of the eye. Blebs also form after trabeculectomies, which is a type of surgery performed to treat glaucoma.

A Bounding Box is an output from a neural network indicating where an object is in an image using a box. While it is typically a box, it can be another shape.

A B-scan is an image composited from a series of A-Scans, by combining each A-Scan with a position and orientation of the transducer at the time the A-Scan was recorded. It is generated by either or both of converting it from a time to a distance using acoustic velocities and by using grayscales, which correspond to A-scan amplitudes, to highlight the features along the A-scan time history trace (the latter also referred to as an A-scan vector).

The bump as referred to herein is the protruding structure located at the intersection of the interface curve and the curve formed by the posterior of the cornea.

The ciliary body is the circumferential tissue inside the eye composed of the ciliary muscle and ciliary processes. There are three sets of ciliary muscles in the eye, the longitudinal, radial, and circular muscles. They are near the front of the eye, above and below the lens. They are attached to the lens by connective tissue called the zonule of Zinn and are responsible for shaping the lens to focus light on the retina. When the ciliary muscle relaxes, it flattens the lens, generally improving the focus for farther objects. When it contracts, the lens becomes more convex, generally improving the focus for closer objects.

The ciliary sulcus is the groove between the iris and ciliary body. The scleral sulcus is a slight groove at the junction of the sclera and cornea.

Fiducial (also referred to herein as fiduciary), means a reference, marker or datum, such as a point or line, in the field of view of an imaging device used as a fixed standard of reference for a fixed basis of comparison or measurement.

Glaucoma is a group of eye conditions that damage the optic nerve, the health of which is vital for good vision. This damage is often caused by an abnormally high pressure in the eye. Glaucoma is one of the leading causes of blindness for older people, and is often linked to a buildup of pressure inside the eye.

Gonioscopy is an exam an ophthalmologist uses to check the angle of an eye.

In this disclosure, grayscale means an image in which the value of each pixel is a single sample representing only intensity information. Images of this sort are composed exclusively of shades of gray, varying from black at the weakest intensity to white at the strongest intensity. Grayscale images are commonly stored with 8 bits per sampled pixel. This pixel depth allows 256 different intensities (shades of gray) to be recorded where grayscale pixels range in values from 0 (black) to 255 (white).

A mask image is an output from a neural network, where each pixel is assigned as either part of a detected object in an image, or background.

In this disclosure, a meridian is defined by the following procedure. In perimetry, the observer's eye is considered to be at the centre of an imaginary sphere. More precisely, the centre of the sphere is in the centre of the pupil of the observer's eye. An observer is looking at a point, the fixation point, on the interior of the sphere. The visual field can be considered to be all parts of the sphere for which the observer can see a particular test stimulus. In perimetric testing, a section of the imaginary sphere is realized as a hemisphere in the centre of which is a fixation point. Test stimuli can be displayed on the hemisphere. To specify loci in the visual field, a polar coordinate system is used, all expressed from the observer's perspective. The origin corresponds to the point on which the observer is fixating. The polar angle is considered to be zero degrees when a locus is horizontally to the right of the fixation point and to increase to a maximum of 360 degrees going anticlockwise. Distance from the origin is given in degrees of visual angle; it's a measure of eccentricity. Each polar axis is a meridian of the visual field. For example, the horizontal meridian runs from the observer's left, through the fixation point, and to the observer's right. The vertical meridian runs from above the observer's line of sight, through the fixation point, and to below the observer's line of sight.

In this disclosure, a moving average (also referred to as a rolling average or running average) is a way of analyzing data points by creating a series of averages of different subsets of adjacent data points in the full data set.

The natural lens (also known as the crystalline lens) is a transparent, biconvex structure in the eye that, along with the cornea, helps to refract light to be focused on the retina. The lens, by changing shape, functions to change the focal distance of the eye so that it can focus on objects at various distances, thus allowing a sharp real image of the object of interest to be formed on the retina. This adjustment of the lens is known as accommodation. The lens is located in the anterior segment of the eye behind the iris.

A neural network (also referred to herein as a machine learning network, artificial network, or network) is a type of AI computer system modeled on the human brain and nervous system. Like a biological neural network (brain), an artificial neural network is composed of artificial neurons or nodes, connected across multiple layers. Each node contains a weight; a positive weight reflects an excitatory connection, while negative values mean inhibitory connections. AI l inputs are modified by a weight and summed. This activity is referred to as a linear combination. Finally, an activation function controls the amplitude of the output. For example, an acceptable range of output is usually between 0 and 1, or it could be −1 and 1. These artificial networks may be used for predictive modeling, adaptive control and applications where they can be trained via a dataset. Self-learning resulting from experience can occur within networks, which can derive conclusions from a complex and seemingly unrelated set of information.

Optical as used herein refers to processes that use light rays.

The optical axis of the eye is a straight line through the centers of curvature of the refracting surfaces of an eye (the anterior and posterior surfaces of the cornea and lens). This is also referred to as on-axis in this document.

A phakic intraocular lens (pIOL) is a special kind of intraocular lens that is implanted surgically into the eye to correct myopia (nearsightedness). It is called “phakic” (meaning “having a lens”) because the eye's natural lens is left untouched. pIOLs are made of clear synthetic plastic. They sit either just in front of, or just behind, the pupil. pIOL implantation is effective in treating high spectacle prescriptions and is widely used to treat younger patients who are not suitable for laser eye surgery. Phakic intraocular lens (phakic IOL or pIOL) implants are an alternative to LASIK and PRK eye surgery for correcting moderate to severe myopia. In some cases, phakic IOLs produce better and more predictable vision outcomes than laser refractive surgery.

Positioner means the mechanism that positions a scan head relative to a selected part of an eye. In the present disclosure, the positioner can move back and forth along the x, y or z axes and rotate in the R direction about the z-axis. In some examples, the positioner does not move during a scan, only the scan head moves. In certain operations, for example, measuring the thickness of a region, the positioner may move during a scan.

Posterior means situated at the back part of a structure; posterior is the opposite of anterior.

The posterior segment comprises the region of the eye from the back of the lens to the rear of the eye comprising the retina and optical nerve.

Refractive means anything pertaining to the focusing of light rays by the various components of the eye, principally the cornea and lens.

ROI means Region of Interest.

Scan head means the mechanism that comprises the ultrasound transducer, the transducer holder and carriage as well as any guide tracks that allow the transducer to be moved relative to the positioner. Guide tracks may be linear, arcuate or any other appropriate geometry. The guide tracks may be rigid or flexible. In some examples, only the scan head is moved during a scan.

The scleral spur in the human eye is an annular structure composed of collagen in the anterior chamber. The scleral spur is a fibrous ring that, on meridional section, appears as a wedge projecting from the inner aspect of the anterior sclera. The spur is attached anteriorly to the trabecular meshwork and posteriorly to the sclera and the longitudinal portion of the ciliary muscle.

Segmentation analysis as used in this disclosure means manipulation of an ultrasound image to determine the boundary or location of an anatomical feature of the eye.

The ciliary sulcus is the groove between the iris and ciliary body. The scleral sulcus is a slight groove at the junction of the sclera and cornea

Schlemm's canal is a circular lymphatic-like vessel in the eye that collects aqueous humor from the anterior chamber and delivers it into the episcleral blood vessels via aqueous veins. Schlemm's canal is a unique vascular structure that functions to maintain fluid homeostasis by draining aqueous humor from the eye into the systemic.

The Schwalbe line is the line formed by the posterior surface of the cornea and delineates the outer limit of the corneal endothelium layer.

Sessile means normally immobile.

The suprachoroid lies between the choroid and the sclera and is composed of closely packed layers of long pigmented processes derived from each tissue.

The suprachoroidalspace is a potential space providing a pathway for uveoscleral outflow and becomes an actual space in choroidal detachment. The hydrostatic pressure in the suprachoroidal space is an important parameter for understanding intraocular fluid dynamics and the mechanism of choroidal detachment.

The trabecular meshwork is an area of tissue in the eye located around the base of the cornea, near the ciliary body, and is responsible for draining the aqueous humor from the eye via the anterior chamber (the chamber on the front of the eye covered by the cornea). The trabecular meshwork plays a very important role in the drainage of aqueous humor. The majority of fluid draining out of the eye is via the trabecular meshwork, then through a structure called Schlemm's canal, into collector channels, then to veins, and eventually back into body's circulatory system.

A trabeculectomy is a type of surgery done for treating glaucoma.

Ultrasonic means sound that is above the human ear's upper frequency limit. When used for imaging an object like the eye, the sound passes through a liquid medium, and its frequency is many orders of magnitude greater than can be detected by the human ear. For high-resolution acoustic imaging in the eye, the frequency is typically in the approximate range of about 5 to about 80 MHz.

An ultrasound scanning device utilizes a transducer capable of sending and/or receiving ultrasonic signals in association with imaging an anatomy.

An ultrasonic arc scanner is an ultrasound scanning device utilizing a transducer that both sends and receives pulses as it moves along 1) an arcuate guide track, which guide track has a center of curvature whose position can be moved to scan different curved surfaces; 2) a linear guide track; and 3) a combination of linear and arcuate guide tracks which can create a range of centers of curvature whose position can be moved to scan different curved surfaces.

The visual axis of the eye is a straight line that passes through both the center of the pupil and the center of the fovea.

Zonules are tension-able ligaments extending from near the outer diameter of the crystalline lens. The zonules attach the lens to the ciliary body which allows the lens to accommodate in response to the action of the ciliary muscle.

Anatomy of the Eye

FIG. 1 illustrates an example 100 of the anatomy of the eye in a region 105 substantially near the iridocorneal angle 107 (also referred to herein as the “angle”) and the scleral spur. The cornea 110, scleral wall 115, and iris 120 all meet in the region 105, with the natural lens 125 (also referred to herein as “lens”) and ciliary body 130 immediately to the right of the location (coordinates) of the union of the cornea 110, scleral wall 115, and iris 120. In some example implementations, the systems and techniques described herein include capturing image data of the region 105. For example, a step in the disclosed techniques described herein includes capturing image data that includes the region 105 of the eye.

FIG. 2 is an example diagram 200 illustrating the angle opening distance (AOD) measured at a location (coordinates) approximately 500 m from the base of the iridocorneal angle 205, at the intersection of the iris and scleral wall. The scleral spur 210 is visible in the example diagram 200. In the example diagram 200, the iridocorneal angle 205 is drawn from the location (coordinates) of the intersection where the scleral wall and iris meet. In some imaging techniques, the intersection of the iris and scleral wall may be difficult to locate due to one or more factors (e.g., the value of the iridocorneal angle 205 (depending on how open the angle is)), and the techniques described herein may utilize the location and/or characteristics (e.g., dimensions) of the scleral spur 210 as the basis for the measurement of the angle opening distance (AOD). The systems and techniques support locating and measuring the scleral spur 210 (and/or other anatomy described herein) using one or more types of imaging technologies (e.g., ultrasound, optical coherence tomography (OCT), etc.).

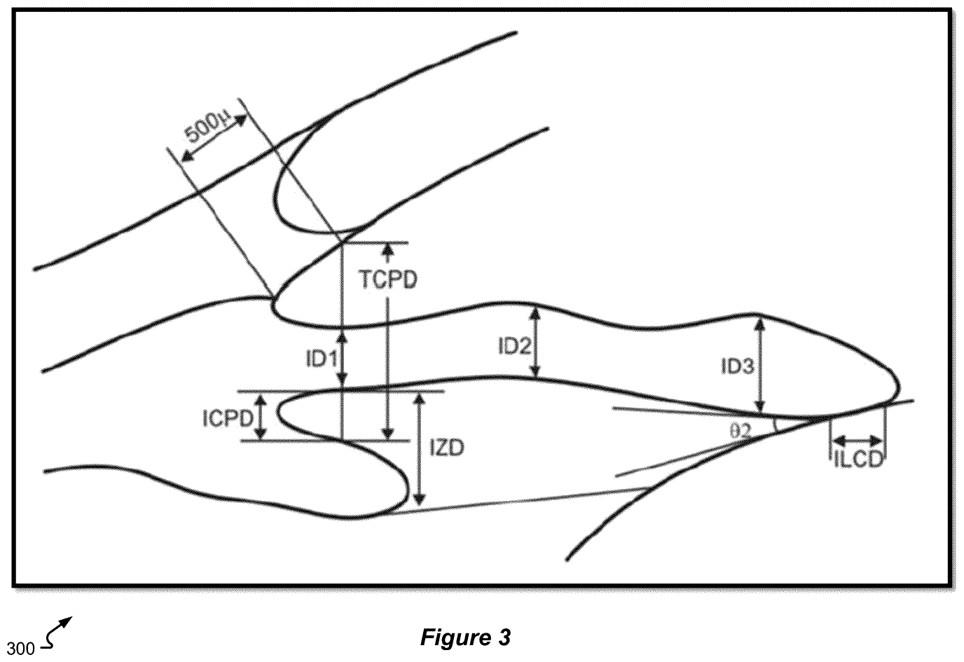

FIG. 3 is an example diagram 300 illustrating other measurements which can be made using the systems and techniques described herein. Example measurements that may be made using the systems and techniques described herein include (and are not limited to):
iris/lens contact distance (ILCD)
iris thickness (ID)
iris zonule distance (IZD)
trabecular ciliary process distance (TCPD)
iris-ciliary process distance (ICPD)
iris-lens angle (ILA)
a measurement associated with an implant in the eye

Aspects of the present disclosure include using imaging techniques described herein in association with measuring ICPD, TCPD, IZD, ILCD, ID1, ID2, ID3, and ILA. In some aspects, utilizing ultrasound technology may support determining the measurements with accuracy and reproducibility. Example aspects of the measurements are discussed in “Anterior Segment Imaging: Ultrasound Biomicroscopy”, Hiroshi Ishikawa, MD* and Joel S. Schuman, MD, Ophthalmol Clin North Am. 7-20, March 2004 which is incorporated herein by reference.

### Acquiring Image Data

Example aspects of the generation of image data in accordance with aspects of the present disclosure are described herein. The image data may be generated or acquired using imaging techniques supported any appropriate device capable of imaging inside the eye. Non-limiting examples of the imaging techniques described herein include ultrasound, OCT, and appropriate imaging techniques used in ophthalmology, and are not limited thereto.

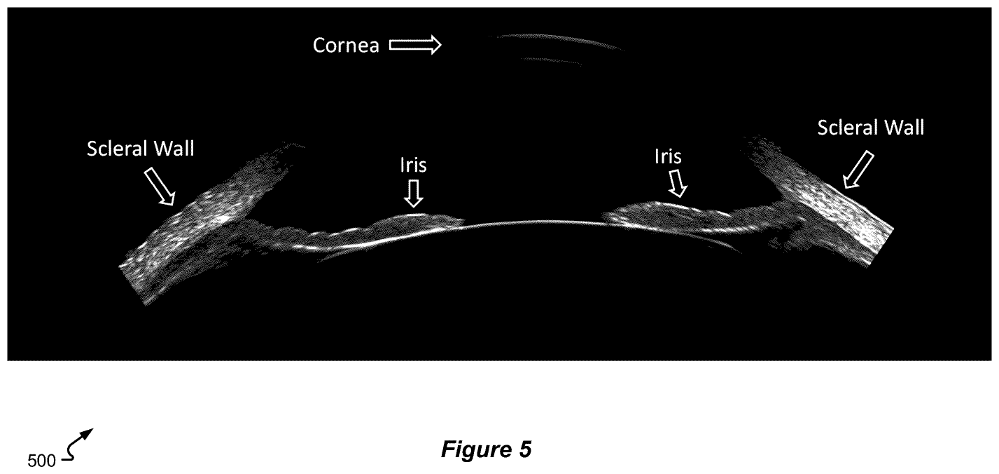

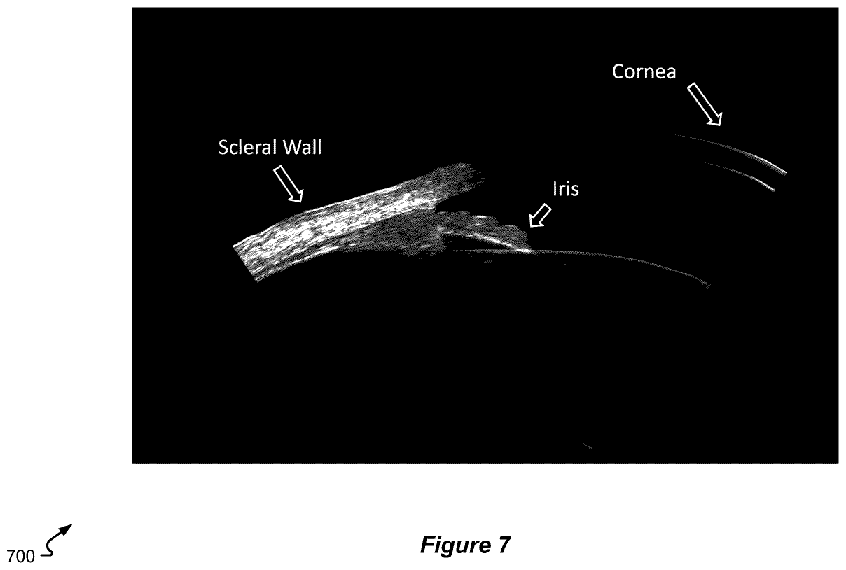

The example images illustrated at FIGS. 5 and 7 were generated using a precision ultrasound device capable of scanning behind the iris, in accordance with aspects of the present disclosure. In some aspects, in accordance with capturing target measurements described herein (e.g., ICPD, TCPD, IZD, ILCD, ID1, ID2, ID3, ILA, a measurement associated with an implant in the eye, etc.), the techniques described herein include generating a complete image of the anterior segment of the eye, including the left and right sides of the scleral/iris region, the anterior cornea to at least mid-lens, and a wide angle sclera to sclera. Example aspects of FIGS. 5 and 7 are later described herein.

### Identifying and Measuring Anatomical Structures Utilizing AI

Aspects of the present disclosure include AI based techniques for locating anatomy within an image. In an example, using captured image data (e.g., once image data has been successfully captured), the systems and techniques include utilizing AI assisted detection to locate anatomy within the image.

In some aspects, the systems and techniques described herein include converting the image (formatting the image/image data) into a format suitable for input into an AI model (also referred to herein as a machine learning model, a neural network model, and the like). For example, the systems and techniques may include converting the image data such that the image size is less than or equal to a target image size. In some example implementations, the target image size may be 512×512 pixels (e.g., the AI models may be capable of processing an input image having an image size less than or equal to 512×512 pixels).

In some aspects, the systems and techniques described herein include converting the image (formatting the image/image data) in accordance with a target shape. In a non-limiting example, the AI models described herein may utilize filters having a square shape. Due to the square shape of the filters present in the model, the systems and techniques described herein may include formatting the image into a square shape using, for example, zero padding (e.g., adding extra rows and columns of zeros to the edges of an image) or other adjustments.

The systems and techniques described herein may be implemented using a range of AI models that support detecting anatomy present in the image data. For example, the AI models may be implemented in a machine learning network, and the output of the machine learning network provides location information about the anatomy present in the image data.

In some example implementations, the systems and techniques may include providing image data to the machine learning network, and the machine learning network may output a mask image or a bounding box in response to processing the image data. The mask image or bounding box may indicate anatomy detected by the machine learning network.

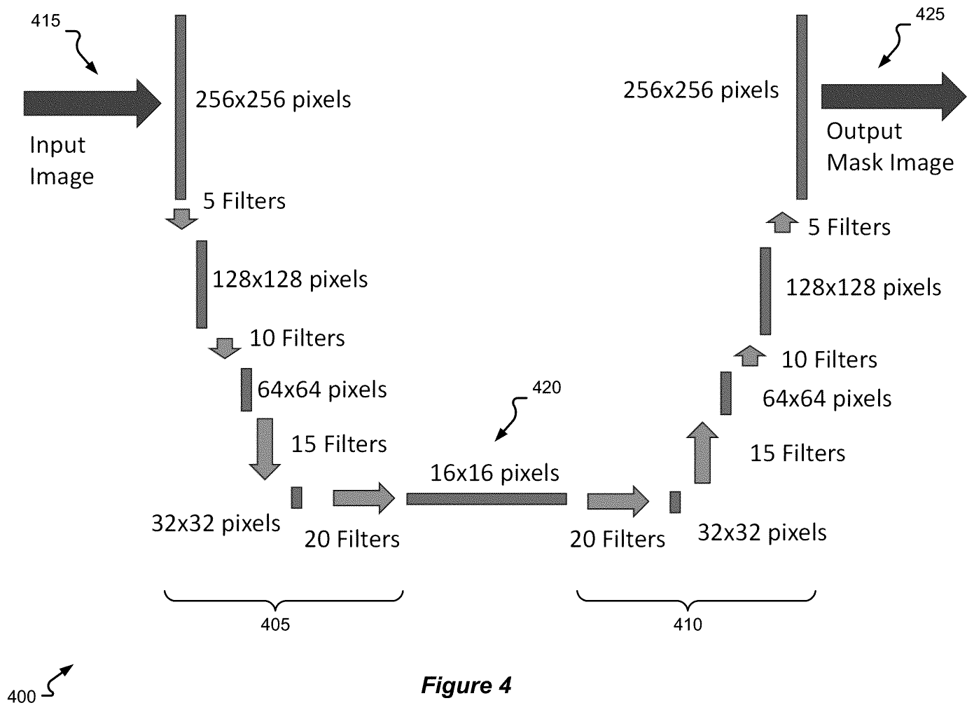

The output from the machine learning network may include location information of the detected anatomy. For example, the systems and techniques described herein may include determining the presence of anatomy in the image data, location information corresponding to the anatomy, and characteristics (e.g., dimensions, etc.) of the anatomy from the mask image and/or bounding box. Example aspects of the AI based techniques are later described with reference to FIG. 4.

FIG. 4 illustrates an example architecture 400 of a neural network that supports of generating a mask image in accordance with aspects of the present disclosure. The neural network may be capable of accepting image data as an input and returning a mask image that identifies the anatomy present in the image.

In the example of FIG. 4, the input to the neural network is a grayscale image of 256×256 pixels, and the output is a mask image of 256×256 pixels. In the mask image output by the neural network, each pixel is categorized as belonging to the background or as a portion of anatomy. Additionally, or alternatively, the neural network may output other indicators (e.g., bounding boxes) that identify the anatomy present in the image.

The neural network may support the detection of any visible anatomy in an input image using an appropriately trained model. Examples of input images (e.g., B-scans) and mask images generated based on the input images, in which the mask images show detected anatomy, are later described with reference to FIGS. 5 through 8.

Non-limiting examples of anatomy detectable by the neural network include:
Cornea
Iris
Scleral wall
Natural lens

The neural network may be a convolutional neural network (CNN) including object detection models. For example, the neural network may utilize convolution to apply filters to images for object detection. Referring to the example of FIG. 4, the neural network may be a modified U-Net, which is a type of convolutional neural network that utilizes convolution to apply filters to images, and the naming of the U-net is due to the U shape of the architecture diagram. In some aspects, object detection models provide increased processing speed and improved results (e.g., increased detection accuracy) compared to less sophisticated models.

The neural network includes an encoder 405 (including encoder filters) and a decoder 410 (including decoder filters). The input image 415 received at the encoder 405 may be an ultrasound image, an infrared image, or the like as supported by aspects of the present disclosure. The encoder 405 accepts the image data of the input image 415 and reduces the image data to an abstracted, highly filtered version of the input data. Accordingly, for example, the encoder 405 outputs an abstracted image 420 (abstracted image data) at a “half-way point.”

This abstracted image 420 output by the encoder 405 is in a format (e.g., image size described herein) appropriate for the decoder 410. The decoder 410 generates a mask image 425 have dimensions (e.g., 256×256 pixel) equal to the dimensions of the input image 415, with pixels categorized as belonging to a portion of anatomy or belonging to the background. In some aspects, the decoder 410 may support categorizing pixels based on anatomy type (e.g., a cornea, a scleral wall, a scleral spur, an iris, a natural lens, a zonule, a ciliary body, a ciliary muscle, and surgically modified tissue, an implant, etc.).

The encoder 405 may include a series of filters. In an example, as the image data moves through the encoder 405, the encoder 405 may apply a series of filters to identify features in the input image 415. In the example of FIG. 4, the filters in the series respectively include 5, 10, 15, and 20 layers. The features identified by filters early in the network are relatively simple compared to the features identified by filters deeper into the network. For example, the filters early in the network support edge detection and/or basic shape recognition, and the filters deeper into the network may have increased complexity. The input image 415 is also reduced in size as the input image 415 progresses further into the network, and the result is a highly abstracted image. The final step in the encoder 405 reduces the input image 415 to a smallest and most abstracted state of the input image 415.

The decoder 410 may generate a mask image 425. For example, the decoder 410 may follow the process as the encoder 405, but in reverse. In an example, the decoder 410 upscales the abstracted image 420 and applies reverse filtering. The final filters of the decoder 410 may categorize (or assign) each of the pixels in the mask image 425 to the background or one of detected pieces of anatomy.

In some aspects, the network may be structured to provide a bounding box as the output. In an example, the network may provide bounding boxes corresponding to detected anatomy or detected portions of anatomy. In some cases, the output of the network may include dimensions of the bounding boxes and categories (e.g., anatomy type) associated with the bounding boxes.

Aspects of the network may include one or more appropriate variations for producing more or less accurate results. The network may be trained or pretrained using training data The quality and quantity of the training data, any pretraining performed on more general image sets, and the like may be selected based on one or more criteria.

In an example, the network may be an untrained network. For example, if training an untrained network, the filters will be initialized with random numbers. The output will be just as random, and the mask image will appear as static. Training the untrained network may include utilizing tens of thousands of labeled images to train the models of the untrained network. Training datasets can come from any imaging device capable of providing imaging data appropriate for the training (e.g., images of sufficient quality for training, images including target anatomy, etc.). For example, images having quality appropriate for training will show at least some of the relevant anatomy without distortion or other anomalies. The image datasets utilized for training may include training and validation sets to ensure that the network may successfully detect target anatomy on images outside the training data set.

Additionally, or alternatively, the network may be a network pretrained (and capable of further training or retraining) on medical images, anatomy, or a wider range of unrelated objects. Implementing such a model for in accordance with aspects of the present disclosure may include modifying the input of the network to accept a greyscale image (e.g., if color is not available) and modifying output layers of the network to classify pixels only to the desired objects. The example training method may be implemented because features (e.g., edges and shapes) present in medical images are also present in other images.

In some cases, the pre-trained network may have been sufficiently trained on an unrelated set of image data, such that the filters may be tuned for detecting anatomy in image data with minimal additional training/refining. For example, such a pre-trained network may be trained/retrained for detecting anatomy in image data using training and validation datasets numbering in the hundreds.

Training data (for training an untrained network or pre-trained network described herein) includes labels with location information to train the models described herein. At least some of the images in both the training and validation sets may include labels corresponding to some or all of the target anatomy, and the training may be implemented using images including some or all of the target anatomy.

During training, whether for an untrained network or a pre-trained network, the output mask image is compared to the labeled image data (the ground truth) of the input image. The difference between the input image and the output mask image is calculated and condensed into a single error value, which is then backpropagated up through the network. Depending on the error value, the weights in each filter in the encoder and decoder are adjusted. A new image is then input to the network, and the cycle repeats.

Training parameters can vary based on target criteria (e.g., target anatomy). In some aspects, the training supported by aspects of the present disclosure may include training the network over a portion of the training dataset, followed by testing the network using the validation set to ensure whether the network is not overfitting to the training data. In some aspects, testing the network using the validation set may include ensuring whether the network can detect objects in image data different from the image data included in the training set. If the error in the validation set is smaller than the prior error value (e.g., related to the ground truth), the network is improving and training may continue. Aspects of the present disclosure include repeating the training as long as training continues to improve the validation result, and there is electricity and computing power available.

In some examples, the systems and techniques described herein may include implementing the training using a graphics processing unit (GPU), as training an advanced model on a large dataset can take weeks on a traditional CPU. The systems and techniques may include running the AI (e.g., trained network) on a CPU and/or a GPU. For example, running the AI on a GPU may provide increases in processing speed.

The following is an example of pre-op anatomy labeled in the training and validation data sets (and detectable using a trained neural network described herein):
1. Cornea
2. Scleral wall
3. Trabecular meshwork
4. Ciliary body
5. Iris
6. Natural lens
7. Zonules
8. Cysts, tumors, and other growths
9. Schlemm's canal and collector channels

The following is an example of post-op anatomy and implants labeled in the training and validation data sets (and detectable using a trained neural network described herein):
1. Bleb
2. Shunt
3. Any MIGS implant, device, or surgical tissue modification
4. Laser ablated tissue

The post-op anatomy and implants may correspond to the type of surgery the patient has undergone or is to undergo.

The examples described herein may support imaging related to Glaucoma applications, but are not limited thereto. The object detection and measurement techniques associated with the anterior segment of the eye as described herein can be used for a wide range of ophthalmic applications. For example, the techniques described herein may utilize a trained AI capable of detecting, and enabling measurement of, the following anatomy/devices:
1. IOL Implant
2. pIOL implant
3. LASIK modifications to the cornea

Other additional measurements capable of being detected and measured in accordance with aspects of the present disclosure include:
1. Anterior chamber depth
2. Angle
3. Anterior chamber width
4. Angle to angle distance
5. Angle to angle lens rise
6. Sulcus to sulcus
7. Sulcus to sulcus lens rise
8. Ciliary body inner radius and/or diameter
9. Vault depth
10. Posterior cornea to anterior pIOL
11. Mid vault width

The network described herein can be used jointly with any device capable of imaging the anterior segment to identify anatomy. As described herein, utilizing the network may support increased processing speed and accuracy associated with identifying anatomy. The techniques described herein include using the detected anatomy and corresponding information (e.g., anatomy location, anatomy characteristics, etc.) to capture a range of measurements relevant to detection and monitoring of a disease (e.g., Glaucoma detection, etc.). The techniques described herein may use the detected anatomy as fiduciaries for measurements that may include additional image processing steps to complete.

FIG. 5 illustrates an example image 500 of the anterior segment of the eye, generated based on imaging signals associated with an imaging device in accordance with aspects of the present disclosure. Image 500 is an example of a complete anterior segment B-scan of the anterior segment of the eye.

In some aspects, the terms “generating an image,” “capturing an image,” and “acquiring an image” may be used interchangeably herein.

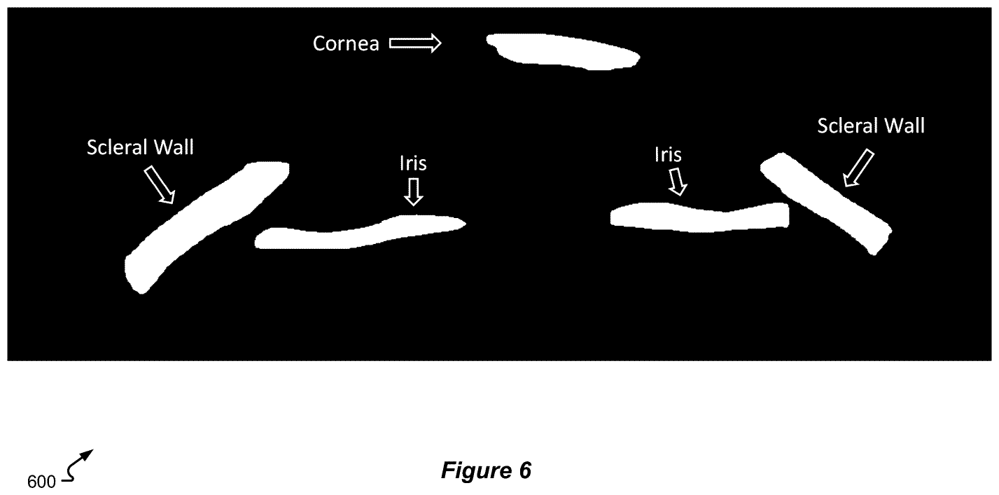

FIG. 6 illustrates an example of a mask image 600 generated based on the image 500 (e.g., B-Scan image) of FIG. 5 using the neural network of FIG. 4, as supported by aspects of the present disclosure. In an example implementation, the systems and techniques support generating the mask image 600 and detecting the cornea, iris, and scleral wall in response to processing (e.g., using the neural network of FIG. 4) the image 500 of FIG. 5. The mask image 600 of FIG. 6 illustrates the detected cornea, iris, and scleral wall.

FIG. 7 illustrates an example image 700 (e.g., a B-scan image) with the optical axis centered above the iridocorneal angle, generated based on imaging signals associated with an imaging device in accordance with aspects of the present disclosure. The image 700 of FIG. 7 supports measurements focused on the iridocorneal angle (e.g., anterior chamber measurements) and other measurements. For example, a larger section of the scleral wall is imaged in the image 700, providing information about the suprachoroid. Based on the image data included in the image 700, the systems and techniques support providing information about the suprachoroidal space (e.g., for cases in which the suprachoroidal space is present). The suprachoroidal space is a potential space between the sclera and choroid that traverses the circumference of the posterior segment of the eye.

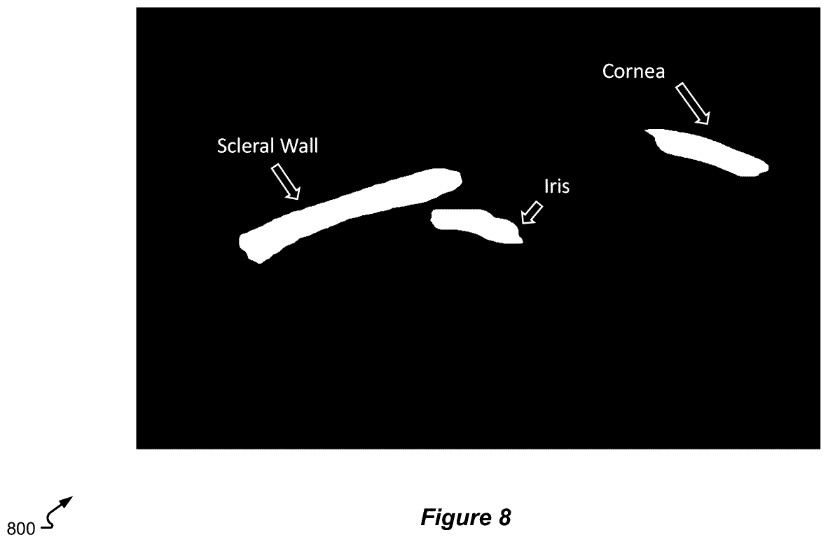

FIG. 8 illustrates an example of mask image 800 generated based on the image 700 (e.g., B-scan image) of FIG. 7 using the neural network of FIG. 4, as supported by aspects of the present disclosure. In an example implementation, the systems and techniques support generating the mask image 800 and detecting the cornea, iris, and scleral wall in response to processing (e.g., using the neural network of FIG. 4) the image 700 of FIG. 7. The mask image 800 of FIG. 8 illustrates the detected cornea, iris, and scleral wall.

### Locating the Scleral Spur to Take Remaining Measurements

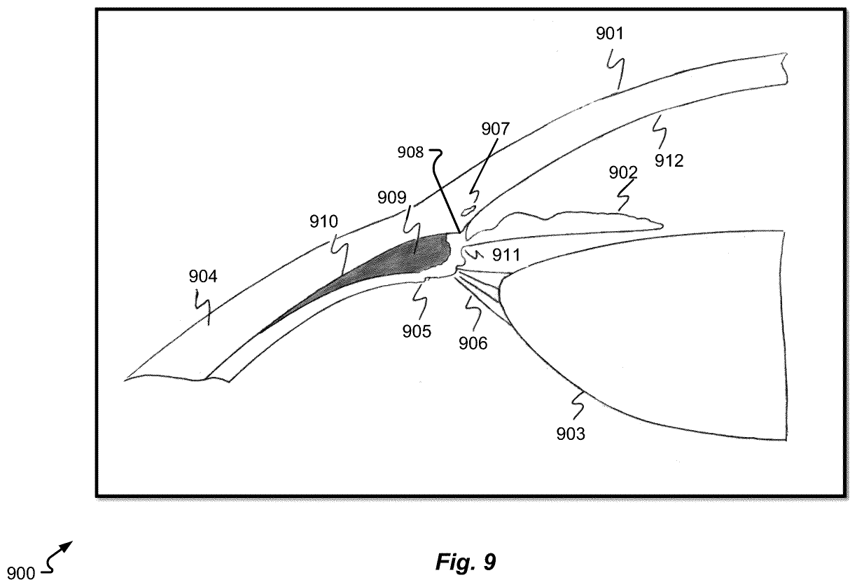

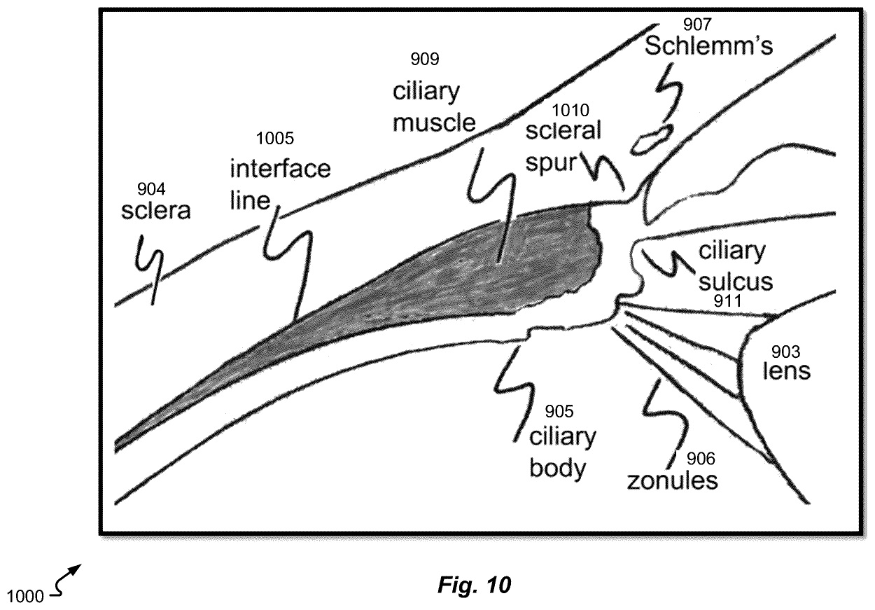

Example aspects of the techniques described herein may include additional processing for certain measurements. For example, the techniques described herein may include using a scleral spur as a fiduciary based on which to take the measurements. In an example, the techniques may include using the scleral wall (as detected by the neural network) as a starting point for determining the scleral spur, as the scleral spur is located along the inner scleral wall and can be identified using the methods described herein. Additionally, the ciliary processes and muscle can be detected using AI techniques supported by the neural network. Examples of features detectable using the AI techniques described herein and examples of features measurable based on the detected features are described with reference to FIGS. 9 and 10.

FIG. 9 is an example diagram 900 of anatomy detectable using techniques supported by aspects of the present disclosure. In the diagram 900, the anatomy is included in the anterior chamber (also referred to herein as anterior segment) of the eye, and one half of the anterior chamber is illustrated. the systems and techniques described support detecting anatomy included in the half of the anterior chamber illustrated in diagram 900 and anatomy included in the opposite half (not illustrated) of the anterior chamber. for example, the opposite half of the of the anterior chamber is a mirror image and includes the same anatomy as the half illustrated in diagram 900.

The diagram 900 illustrates geometric structures based on which the systems and techniques described herein support detecting one or more target structures (e.g., a scleral spur, etc.) described herein. The cornea 901, the iris 902, the lens 903, the sclera 904, and the ciliary body 905 are illustrated in the example diagram 900. The ciliary body 905 includes the ciliary muscle 909.

The ciliary sulcus 911 is illustrated between the iris 902 and the ciliary body 905. Zonules 906 and Schlemm's canal 907 are also illustrated for reference. The interface curve 910 is formed by the interface between the sclera 904 and the ciliary muscle 909. A line projected from a point on the interface curve 910 at the local slope is referred to herein as a “scleral slope line”.

Interface curve 910 intersects Schwalbe line 912, and the protruding structure located at the intersection is called the bump 908. The Schwalbe line 912 is the curve formed by the posterior of the cornea 901.

FIG. 10 further illustrates the interface line 1005 between the sclera 904 and ciliary muscle 909. A suprachoroidal space can appear at or near the interface line 1005 following some Glaucoma surgeries. The interface line 1000 as illustrated in FIG. 10 is illustrated as a boundary between the sclera 904 (relatively lighter) and the ciliary muscle 909 (relatively darker). FIG. 10 illustrates an example location of a scleral spur 1010.

Aspects of the present disclosure support locating the scleral spur using one or more of the following methods:
1. The Ciliary Muscle method.
2. First variation of the Bump method.
3. Second variation of the Bump method.
4. Third variation of the Bump method.
5. Schwalbe Line method.

A general description of the methods 1 through 5 can be found in “The Effect of Scleral Spur Identification Methods on Structural Measurements by Anterior Segment Optical Coherence Tomography” Seager, Wang, Arora, Quigley, Journal of Glaucoma, Vol. 23, No 1, January 2014, which is incorporated herein by reference.

In some aspects, the systems and techniques described herein support using several of the methods for locating the scleral spur and providing a predicted location of the scleral spur based on a comparison of the results of the methods. For example, the systems and techniques described herein may include comparing the locations of the potential spurs determined using the four methods above. The systems and techniques may include determining the location of the scleral spur based on predictions of the scleral spur location as provided the one or more methods. For example, the systems and techniques may include considering proximity of the predictions of the scleral spur location to each other. In another example, the systems and techniques may include considering proximity of the predictions of the scleral spur location to the iris root.

The systems and techniques may include calculating a confidence score or confidence factor associated with the scleral spur location based on the described factors. In some aspects, the systems and techniques may include repeatedly calculating the location of the spur until a target accuracy associated with the calculated location is reached. For example, the systems and techniques may repeatedly calculate the location of the scleral spur until the confidence score or confidence factor is equal to a threshold value (e.g., a target score, a target confidence factor, etc.).

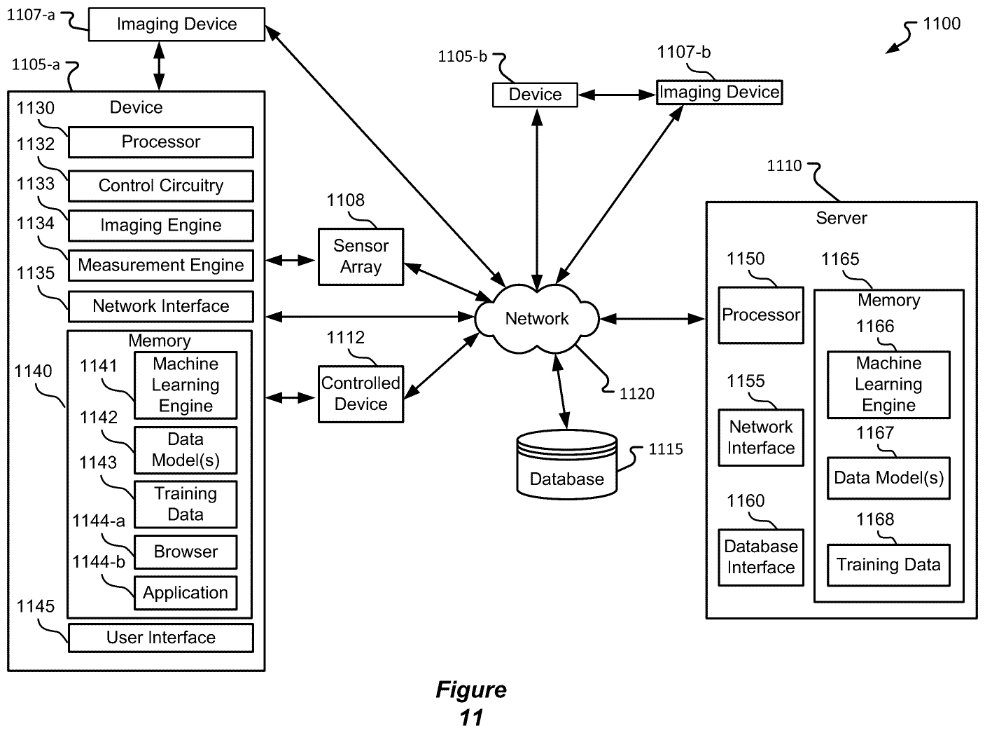

Once the scleral spur has been located, one or more measurements described herein based on the location and/or characteristics (e.g., dimensions) of the scleral spur can be made. Example aspects of steps associated with the described anatomy detection (e.g., of the scleral spur) and measurements are later described with reference to FIG. 11.

As described herein, according to some example implementations, techniques are disclosed for capturing image data of the human eye using an ophthalmic imaging device, utilizing artificial intelligence trained on a labeled dataset to locate anatomy within the image data and, using the detected anatomy as a fiduciary, taking measurements of the eye relevant to the detection and monitoring of a disease (e.g., Glaucoma). In some cases, over time, the measurements can change and can indicate a change, or be a precursor for a change, of intraocular pressure (IOP).

FIG. 11 illustrates an example of a system 1100 supportive of the techniques described herein in accordance with aspects of the present disclosure. The system 1100 may include a device 1105 (e.g., device 1105-a, device 1105-b) electrically coupled to an imaging device 1107 (e.g., imaging device 1107-a, imaging device 1107-b). In some example implementations the device 1105 may be integrated with the imaging device 1107. The system 1100 may be referred to as a control and signal processing system.

The device 1105 may support data processing (e.g., image processing), control operations, object detection (e.g., detecting or locating one or more target structures included in the eye), disease identification or prediction (e.g., determining a presence, an absence, a progression, or a stage of a disease of the eye based on one or more measurements associated with the eye), and communication in accordance with aspects of the present disclosure. The device 1105 may be a computing device. In some aspects, the device 1105 may be a wireless communication device. Non-limiting examples of the device 1105 may include, for example, personal computing devices or mobile computing devices (e.g., laptop computers, mobile phones, smart phones, smart devices, wearable devices, tablets, etc.). In some examples, the device 1105 may be operable by or carried by a human user. In some aspects, the device 1105 may perform one or more operations autonomously or in combination with an input by the user, the device 1105, and/or the server 1110.

The imaging device 1107 may support transmitting and/or receiving any suitable imaging signals in association with acquiring or generating image data described herein of an anatomical feature (e.g., eye, tissue, an implant, etc.) of a patient. For example, the image data may include an A-scan, B-scan, ultrasound image data, infrared image data (also referred to herein as thermal image data), or the like.

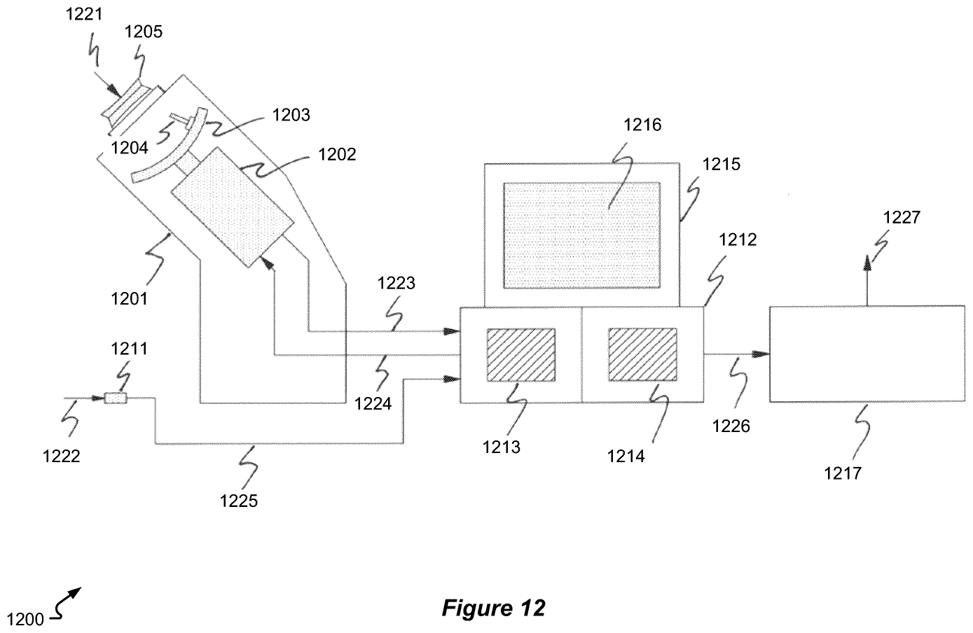

In an example, the imaging signals may include ultrasound signals, and the imaging device 1107 may transmit and/or receive ultrasound pulses in association with acquiring or generating the image data. In another example, the imaging signals may include infrared laser light transmitted and/or received in association with acquiring or generating the image data. A non-limiting example of the imaging device 1107 includes an arc scanning machine 1201 later described with reference to FIG. 12. In some aspects, the imaging device 1107 includes a sensor array 1108 and a controlled device 1112.

The sensor array 1108 includes linear or angular position sensors that, among other things, track the relative and/or absolute positions of the various movable components and the alignment of various stationary and moveable components, such as, but not limited to, the one or more position tracking sensors, the positioning arms and probe carriage assembly, the fixation lights, the optical video camera, the arcuate guide assembly, the transducer probes, the probe carriage, the linear guide track, the motors to move the position arms, motors to move the arcuate guide assembly, and motors to move the probe carriage. The sensor array 1108 may include any suitable type of positional sensors, including inductive non-contact position sensors, string potentiometers, linear variable differential transformers, potentiometers, capacitive transducers, eddy-current sensors, Hall effect sensors, proximity sensors (optical), grating sensors, optical encoders (rotary or linear), and photo diode arrays. Candidate sensor types which may be included in the sensory array 1108 are discussed in U.S. Pat. No. 8,1158,252, example aspects of which are incorporated herein by reference.

The controlled device 1112 is any device having an operation or feature controlled by the device 1105. Controlled devices include the various movable or activatable components, such as, but not limited to, the one or more position tracking sensors, the positioning arms, the transducer carriage assembly, the fixation lights, the optical video camera, the arcuate guide assembly, the transducer probes, the probe carriage, the linear guide track, the motors to move the position arms, motors to move the arcuate guide assembly, and motors to move the probe carriage.

The system 1100 may include a server 1110, a database 1115, and a communication network 1120. The server 1110 may be, for example, a cloud-based server. In some aspects, the server 1110 may be a local server connected to the same network (e.g., LAN, WAN) associated with the device 1105. The database 1115 may be, for example, a cloud-based database. In some aspects, the database 1115 may be a local database connected to the same network (e.g., LAN, WAN) associated with the device 1105 and/or the server 1110. The database 1115 may be supportive of data analytics, machine learning, and AI processing.

The communication network 1120 may facilitate machine-to-machine communications between any of the device 1105 (or multiple devices 1105), the server 1110, or one or more databases (e.g., database 1115). The communication network 1120 may include any type of known communication medium or collection of communication media and may use any type of protocols to transport messages between endpoints. The communication network 1120 may include wired communications technologies, wireless communications technologies, or any combination thereof.

The Internet is an example of the communication network 1120 that constitutes an Internet Protocol (IP) network consisting of multiple computers, computing networks, and other communication devices located in multiple locations, and components in the communication network 1120 (e.g., computers, computing networks, communication devices) may be connected through one or more telephone systems and other means. Other examples of the communication network 1120 may include, without limitation, a standard Plain Old Telephone System (POTS), an Integrated Services Digital Network (ISDN), the Public Switched Telephone Network (PSTN), a Local Area Network (LAN), a Wide Area Network (WAN), a wireless LAN (WLAN), a Session Initiation Protocol (SIP) network, a Voice over Internet Protocol (VoIP) network, a cellular network, and any other type of packet-switched or circuit-switched network known in the art. In some cases, the communication network 1120 may include of any combination of networks or network types. In some aspects, the communication network 1120 may include any combination of communication mediums such as coaxial cable, copper cable/wire, fiber-optic cable, or antennas for communicating data (e.g., transmitting/receiving data).

In various aspects, settings, configurations, and operations of the any of the devices 1105, the imaging devices 1107, the server 1110, database 1115, and the communication network 1120, may be configured and modified by any user and/or administrator of the system 1100.

Aspects of the devices 1105 and the server 1110 are further described herein. A device 1105 (e.g., device 1105-a) may include a processor 1130, control circuitry 1132, imaging engine 1133, measurement engine 1134, a network interface 1135, a memory 1140, and a user interface 1145. In some examples, components of the device 1105 (e.g., processor 1130, network interface 1135, memory 1140, user interface 1145) may communicate over a system bus (e.g., control busses, address busses, data busses) included in the device 1105. In some cases, the device 1105 may be referred to as a computing resource.

The processor 1130 may include processing circuitry supportive of the techniques described herein.

The control circuitry 1132 may be capable of controlling (e.g., via control signals) features of one or more imaging devices 1107. The control circuitry 1132 (also referred to herein as a controller) may receive and process positioning signals from the sensor array 1108 and generate and transmit appropriate commands to the monitored controlled device 1112.

In one or more embodiments, the control circuitry 1132 determines an adjustment to the position of the transducer and/or the OCT sample arm probe and the OCT reference arm based on receiving a control measurement input from the sensor array 1108. In one or more embodiments, the control circuitry 1132 provides a control input to the drive mechanism of the probe carriage, the positioning arm, the arcuate guide assembly, and/or the linear guide track. In one or more embodiments, the control circuitry 1132 provides a control input to include controlling the power, frequency, signal/noise ratio, pulse rate, gain schedule, saturation thresholds, and sensitivity of the optical and/or ultrasound transducers. In one or more embodiments, the control circuitry 1132 utilizes control algorithms including at least one of on/off control, proportional control, differential control, integral control, state estimation, adaptive control and stochastic signal processing. Control circuitry 1132 may monitor and determine if any faults or diagnostic flags have been identified in one or more elements, such as the optical and/or ultrasound transducers and/or carriage.

Imaging engine 1133 (also referred to herein as an ultrasound B-scan imaging module) may support receiving and processing A-scan images and B-scan images to produce two-, three-, or four-dimensional images of target ocular components or features.

Measurement engine 1134 (also referred to herein as glaucoma measurement module) may support determining, as discussed herein, the dimensions and positional relationships of selected ocular components and/or features associated with the onset of glaucoma and tracking the progression of glaucoma.

In some non-limiting examples, the system 1100 may support determining points of interest (e.g., a target structure described herein, for example, a scleral spur, as a fiduciary) and measurements described herein based on the points of interest. In some aspects, the measurements may include points/measurements posterior to and anterior to the iris (in front of and behind the iris). In some aspects, using the imaging device 1107 (e.g., an ultrasound arc scanning device), the system 1100 may form a B-scan image of the anterior segment (anterior cornea to approximately mid lens, wide angle sclera to sclera) including the left and right sides of the scleral/iris region. The system 1100 supports determining/locating other example target structures and determining other example measurements described herein.

In some cases, the device 1105 may transmit or receive packets to one or more other devices (e.g., another device 1105, an imaging device 1107, the server 1110, the database 1115) via the communication network 1120, using the network interface 1135. The network interface 1135 may include, for example, any combination of network interface cards (NICs), network ports, associated drivers, or the like. Communications between components (e.g., processor 1130, memory 1140) of the device 1105 and one or more other devices (e.g., another device 1105, an imaging device 1107, the database 1115) connected to the communication network 1120 may, for example, flow through the network interface 1135.

The processor 1130 may correspond to one or many computer processing devices. For example, the processor 1130 may include a silicon chip, such as a FPGA, an ASIC, any other type of IC chip, a collection of IC chips, or the like. In some aspects, the processors may include a microprocessor, CPU, a GPU, or plurality of microprocessors configured to execute the instructions sets stored in a corresponding memory (e.g., memory 1140 of the device 1105). For example, upon executing the instruction sets stored in memory 1140, the processor 1130 may enable or perform one or more functions of the device 1105.

The memory 1140 may include one or multiple computer memory devices. The memory 1140 may include, for example, Random Access Memory (RAM) devices, Read Only Memory (ROM) devices, flash memory devices, magnetic disk storage media, optical storage media, solid-state storage devices, core memory, buffer memory devices, combinations thereof, and the like. The memory 1140, in some examples, may correspond to a computer-readable storage media. In some aspects, the memory 1140 may be internal or external to the device 1105.

The processor 1130 may utilize data stored in the memory 1140 as a neural network (also referred to herein as a machine learning network). The neural network may include a machine learning architecture. The neural network may support machine learning (artificial intelligence) techniques described herein.

In some aspects, the neural network may be or include an artificial neural network (ANN). In some other aspects, the neural network may be or include any appropriate machine learning network such as, for example, a deep learning network, a convolutional neural network, or the like. Some elements stored in memory 1140 may be described as or referred to as instructions or instruction sets, and some functions of the device 1105 may be implemented using machine learning techniques.

The memory 1140 may be configured to store instruction sets, neural networks, and other data structures (e.g., depicted herein) in addition to temporarily storing data for the processor 1130 to execute various types of routines or functions. For example, the memory 1140 may be configured to store program instructions (instruction sets) that are executable by the processor 1130 and provide functionality of machine learning engine 1141 described herein. The memory 1140 may also be configured to store data or information that is useable or capable of being called by the instructions stored in memory 1140. One example of data that may be stored in memory 1140 for use by components thereof is a data model(s) 1142 (e.g., a neural network model (also referred to herein as a machine learning model) or other model described herein) and/or training data 1143 (also referred to herein as a training data and feedback).

The machine learning engine 1141 may include a single or multiple engines. The device 1105 (e.g., the machine learning engine 1141) may utilize one or more data models 1142 for recognizing and processing information obtained from one or more imaging devices 1107, other devices 1105, the server 1110, and the database 1115. In some aspects, the device 1105 (e.g., the machine learning engine 1141) may update one or more data models 1142 based on learned information included in the training data 1143. In some aspects, the machine learning engine 1141 and the data models 1142 may support forward learning based on the training data 1143. The machine learning engine 1141 may have access to and use one or more data models 1142.

The data model(s) 1142 may be built and updated by the machine learning engine 1141 based on the training data 1143. The data model(s) 1142 may be provided in any number of formats or forms. Non-limiting examples of the data model(s) 1142 include Decision Trees, Support Vector Machines (SVMs), Nearest Neighbor, and/or Bayesian classifiers. In some aspects, the data model(s) 1142 may include a predictive model such as an autoregressive model. Other example aspects of the data model(s) 1142, such as generating (e.g., building, training) and applying the data model(s) 1142, are described with reference to the figure descriptions herein. The data model(s) 1142 may include aspects of machine learning models described herein.

The machine learning engine 1141 and model(s) 1142 may implement example aspects of the machine learning methods and learned functions described herein. Data within the database of the memory 1140 may be updated, revised, edited, or deleted by the machine learning engine 1141.

The device 1105 may render a presentation (e.g., visually, audibly, using haptic feedback, etc.) of an application 1144 (e.g., a browser application 1144-a, an application 1144-b). The application 1144-b may be an application associated with controlling features of an imaging device 1107 as described herein. For example, the application 1144-b may enable control of the device 1105 and/or an imaging device 1107 described herein.

In an example, the device 1105 may render the presentation via the user interface 1145. The user interface 1145 may include, for example, a display (e.g., a touchscreen display), an audio output device (e.g., a speaker, a headphone connector), or any combination thereof. In some aspects, the applications 1144 may be stored on the memory 1140. In some cases, the applications 1144 may include cloud-based applications or server-based applications (e.g., supported and/or hosted by the database 1115 or the server 1110). Settings of the user interface 1145 may be partially or entirely customizable and may be managed by one or more users, by automatic processing, and/or by artificial intelligence.

In an example, any of the applications 1144 (e.g., browser application 1144-a, application 1144-b) may be configured to receive data in an electronic format and present content of data via the user interface 1145. For example, the applications 1144 may receive data from an imaging device 1107, another device 1105, the server 1110, and/or the database 1115 via the communication network 1120, and the device 1105 may display the content via the user interface 1145.

The database 1115 may include a relational database, a centralized database, a distributed database, an operational database, a hierarchical database, a network database, an object-oriented database, a graph database, a NoSQL (non-relational) database, etc. In some aspects, the database 1115 may store and provide access to, for example, any of the stored data described herein.

The server 1110 may include a processor 1150, a network interface 1155, database interface instructions 1160, and a memory 1165. In some examples, components of the server 1110 (e.g., processor 1150, network interface 1155, database interface 1160, memory 1165) may communicate over a system bus (e.g., control busses, address busses, data busses) included in the server 1110. The processor 1150, network interface 1155, and memory 1165 of the server 1110 may include examples of aspects of the processor 1130, network interface 1135, and memory 1140 of the device 1105 described herein.

For example, the processor 1150 may be configured to execute instruction sets stored in memory 1165, upon which the processor 1150 may enable or perform one or more functions of the server 1110. In some examples, the server 1110 may transmit or receive packets to one or more other devices (e.g., a device 1105, the database 1115, another server 1110) via the communication network 1120, using the network interface 1155.

Communications between components (e.g., processor 1150, memory 1165) of the server 1110 and one or more other devices (e.g., a device 1105, the database 1115, etc.) connected to the communication network 1120 may, for example, flow through the network interface 1155.

In some examples, the database interface instructions 1160 (also referred to herein as database interface 1160), when executed by the processor 1150, may enable the server 1110 to send data to and receive data from the database 1115. For example, the database interface instructions 1160, when executed by the processor 1150, may enable the server 1110 to generate database queries, provide one or more interfaces for system administrators to define database queries, transmit database queries to one or more databases (e.g., database 1115), receive responses to database queries, access data associated with the database queries, and format responses received from the databases for processing by other components of the server 1110.

The memory 1165 may be configured to store instruction sets, neural networks, and other data structures (e.g., depicted herein) in addition to temporarily storing data for the processor 1150 to execute various types of routines or functions. For example, the memory 1165 may be configured to store program instructions (instruction sets) that are executable by the processor 1150 and provide functionality of a machine learning engine 1166. One example of data that may be stored in memory 1165 for use by components thereof is a data model(s) 1167 (e.g., any data model described herein, a neural network model, etc.) and/or training data 1168.

The data model(s) 1167 and the training data 1168 may include examples of aspects of the data model(s) 1142 and the training data 1143 described with reference to the device 1105. The machine learning engine 1166 may include examples of aspects of the machine learning engine 1141 described with reference to the device 1105. For example, the server 1110 (e.g., the machine learning engine 1166) may utilize one or more data models 1167 for recognizing and processing information obtained from imaging devices 1107, devices 1105, another server 1110, and/or the database 1115. In some aspects, the server 1110 (e.g., the machine learning engine 1166) may update one or more data models 1167 based on learned information included in the training data 1168.

In some aspects, components of the machine learning engine 1166 may be provided in a separate machine learning engine in communication with the server 1110.

The data model(s) 1142 may support locating one or more target structures (e.g., tissue, surgically modified tissue, pharmacologically modified tissue, an implant, etc.) included in the eye as described herein. For example, the data model(s) 1142 may support detecting and locating one or more target structures included in the eye, without human intervention. The data model(s) 1142 may support determining a presence, an absence, a progression, or a stage of a disease of the eye as described herein. For example, the data model(s) 1142 may support determining a presence, an absence, a progression, or a stage of a disease of the eye based on one or more measurements associated with an anterior portion of the eye, without human intervention.

Aspects of the present disclosure may support machine learning techniques for building and/or training a data model(s) 1142. The data model(s) 1142 may include untrained models and/or pre-trained models. In an example, the data model(s) 1142 may be trained or may learn during a training phase associated with locating one or more target structures included in the eye. In another example, the data model(s) 1142 may be trained or may learn during a training phase associated with determining a presence, an absence, a progression, or a stage of a disease of the eye based on measurements associated with an anterior portion of the eye. In some aspects, the data a

FIG. 12 illustrates an example apparatus 1200 in accordance with aspects of the present disclosure. In the example described herein, apparatus 1200 may include arc scanning machine 1201 and computer 1212, in which arc scanning machine 1201 and computer 1212 are electrically coupled and integrated in a common housing. In some other aspects, the features described with reference to FIG. 12 may be implemented as a system in which arc scanning machine 1201 and computer 1212 are standalone components electrically coupled and/or wirelessly coupled (e.g., via network 1120 of FIG. 11).

FIG. 12 is a schematic representation of the control functions of the apparatus 1200. The apparatus 1200 includes an arc scanning machine 1201 which includes an arc guide positioning mechanism 1202 (also referred to herein as positioning head 1202), an arc guide (or arcuate guide or arc track) 1203, an ultrasonic transducer 1204 and a disposable eyepiece 1205. The apparatus 1200 may also include a scan head in which an arcuate guide track is mounted on a linear guide track.

The arc scanning machine 1201 is electrically coupled to a computer 1212 which includes a processor module 1213, a memory module 1214, and a video monitor 1215 including a video screen 1216. The computer 1212 is connected to and may receive inputs via one or more operator input peripherals 1211 (e.g., a mouse device, a keyboard (not shown), speech recognition device, etc.). The computer 1212 is also connected to one or more output devices (e.g., a printer 1217, a network interface card 1218, etc.).

The patient is seated at the machine 1201 with one of their eyes engaged with disposable eyepiece 1205. The patient's eye component to be imaged is represented by input 1221. The operator, using an input peripheral 1211, inputs information into computer 1212 selecting the type of scan and scan configurations as well as the desired type of output image and analyses. The operator, using input peripheral 1211, a video camera in scanning machine 1201, and video screen 1216, may center a set of cross hairs displayed on video screen 1216 on the desired component of the patient's eye, also displayed on video screen 1216, setting one of the cross hairs as the prime meridian for scanning.

Once the prime meridian has been set, the operator may instruct computer 1212 using input peripheral 1211 to proceed with the scanning sequence. In response to the user input, the computer processor 1213 may execute stored instructions in association with the procedure. For example, the computer 1212 may issue instructions via path 1224 to the positioning head 1202, the arcuate track 1203, and a transducer carriage and receives positional and imaging data via path 1223. The computer 1212 may store the positional and imaging data in memory module 1214.

In an example implementation, the computer processor 1213 may proceed with the example sequence of operations: (1) rough focus transducer 1204 on the selected eye component; (2) accurately center arcuate track 1203 with respect to the selected eye component; (3) accurately focus transducer 1204 on the selected feature of the selected eye component; (4) rotate the arcuate track through a substantial angle and repeat steps (1) through (3) on a second meridian; (5) rotate the arcuate track back to the prime meridian; (6) initiate a set of A-scans along each of selected scan meridians, storing image data associated with the A-scans in memory module 1214; (7) utilizing processor 1213, converting the A-scans for each meridian into a set of B-scans and then processing the B-scans to form an image associated with each meridian; (8) performing one or more selected analyses on the A-scans, B-scans, and images associated with each or all of the meridians scanned; and (9) outputting the data 1226 in a preselected format to an output device 1217 (e.g., a printer, a network interface card for transmission over the network 1120). In some aspects, the computer 1212 may store the output in memory module 1214 for later retrieval on video screen 1216. Additionally, or alternatively, the computer 1212 may transmit the output to remote computers or other output devices via any number of appropriate data transmission techniques.

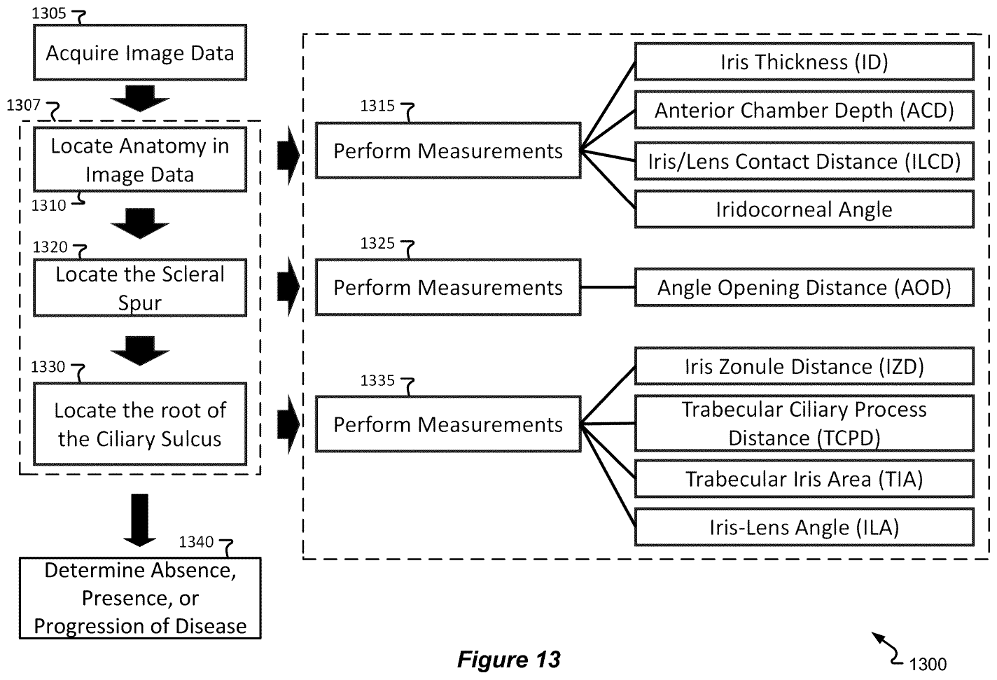

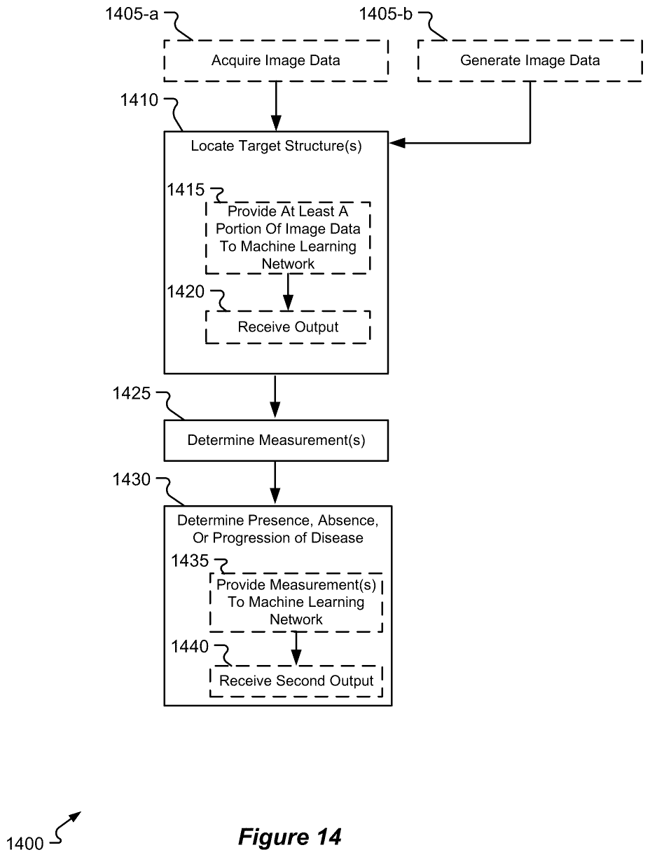

FIG. 13 and FIG. 14 illustrate example process flows 1300 and 1400 that support aspects of the present disclosure. In some examples, process flows 1300 and 1400 may be implemented by aspects of system 1100 described with reference to FIG. 11. Further, process flows 1300 and 1400 may be implemented by a device 1105 and/or a server 1110 described with reference to FIG. 11.

In the following description of the process flows 1300 and 1400, the operations may be performed in a different order than the order shown, or the operations may be performed in different orders or at different times. Certain operations may also be left out of the process flows 1300 and 1400, or other operations may be added to the process flows 1300 and 1400. It is to be understood that while a device 1105 is described as performing a number of the operations of process flows 1300 and 1400, any device (e.g., another device 1105 in communication with the device 1105, another server 1110 in communication with the server 1110) may perform the operations shown.

The process flows 1300 and 1400 may be implemented by an apparatus including: a processor; and memory (e.g., a non-transitory computer readable storage medium) in electronic communication with the processor, wherein instructions stored in the memory are executable by the processor to perform one or more operations of the process flows 1300 and 1400.

Referring to FIG. 13, the process flow 1300 supports automatically generating an image (e.g., a B-Scan, etc.), utilizing AI to detect anatomy in the image, and creating measurements based on the detected anatomy in accordance with aspects of the present disclosure.

At 1305, the process flow 1300 may include acquiring image data of an eye of a patient. In an example, the process flow 1300 may include acquiring the image data based on one or more imaging signals emitted by an imaging device 1107 described herein. In another example, the image data may be pre-acquired image data stored at, for example, database 1115.

In an example, at 1305, the process flow 1300 may include acquiring image data from a PACS/DICOM type system. PACS is a system that is used to manage and store medical images and other clinical data, and DICOM is a standard that is used to format and transmit the images and data in a way that is compatible with different systems and devices. In the DICOM standard, images and the pixel dimensions are provided, and the systems and techniques support providing analysis described herein based on the images and pixel dimensions.

In some aspects, the image data may include a single image of the eye of the patient or multiple images of the eye.

At 1307, the process flow 1300 may include processing the image data and/or location data associated with one or more target structures (e.g., patient anatomy) detected in the image data.

In an example, at 1310, the process flow 1300 may include locating one or more target structures (e.g., patient anatomy) in the image data of the eye. In some examples, the one or more target structure may include tissue included in the eye, surgically modified tissue included in the eye, pharmacologically modified tissue included in the eye, an implant included in the eye, and the like. Non-limiting examples of the target structures include the cornea, iris, natural lens, and scleral wall of the eye, and are not limited thereto.

The process flow 1300 may include locating the one or more target structures using one or more machine learning techniques (e.g., machine learning models, artificial intelligence, etc.) described herein. The output provided using the one or more machine learning techniques may be referred to as AI detected locations of the target structures. For example, aspects of the present disclosure described herein in association with locating anatomy (as described with reference to 1310, 1320, and 1330) may include generating predictions of locations of a target structure in combination with probability scores and/or confidence scores associated with the predictions. The techniques described herein may include outputting a location of a target structure for cases in which a corresponding probability score and/or confidence score is equal to or greater than a threshold value.

In one or more example implementations, at 1310, the process flow 1300 may include locating all anatomy present in the image data (e.g., in the image or images). For example, at 1310, the process flow 1300 may include locating the cornea, iris, natural lens, and scleral wall of the eye.

At 1315, the process flow 1300 may include performing measurements associated with the eye of the patient based on the anatomy located at 1310.

For example, using the AI detected location of the Iris, the process flow 1300 may include measuring the iris thickness (ID). In another example, using the AI determined positions of the natural lens and cornea, the process flow 1300 may include measuring the anterior chamber depth (ACD). In some other examples, using the AI determined positions of the natural lens and iris, the process flow 1300 may include determining the iris/lens contact distance (ILCD). In another example, using the AI determined locations of the iris and scleral wall, the process flow 1300 may include locating and/or measuring the iridocorneal angle.

At 1320, the process flow 1300 may include locating the scleral spur of the eye based on the AI determined locations of the iris and scleral wall. For example, using the AI determined locations of the iris and scleral wall, the process flow 1300 may include locating the scleral spur along the inner surface of the scleral wall, at a location within a threshold distance of the iridocorneal angle.

At 1325, the process flow 1300 may include performing measurements associated with the eye of the patient based on one or more measurements of 1315, the location of the scleral spur (as determined at 1320), characteristics (e.g., location information, one or more dimensions, etc.) of the scleral spur, and/or characteristics of the iridocorneal angle (e.g., apex of the iridocorneal angle (also referred to herein as the close of the angle)).

For example, at 1325, the process flow 1300 may include calculating the angle opening distance (AOD). The process flow 1300 may include calculating the angle opening distance (AOD) at a position (e.g., coordinates) located 500 microns or about 500 microns from the close (e.g., at the apex) of the iridocorneal angle. In some aspects, the process flow 1300 may include calculating the angle opening distance (AOD) at a position located a target distance (e.g., a distance ranging from about 0 microns to about 1000 microns) from the close (e.g., at the apex) of the iridocorneal angle or the scleral spur, depending on the analysis being performed.

At 1330, the process flow 1300 may include locating the root of the ciliary sulcus (also referred to herein as the iris root). For example, using the AI determined position of the iris (as determined at 1310), the process flow 1300 may include locating the root of the ciliary sulcus.

At 1335, the process flow 1300 may include performing one or more measurements using one or more of the target structures (e.g., as located at 1310, 1320, or 1330) as a fiduciary. For example, the process flow 1300 may include performing the one or more measurements based on proximity of a target structure to the root of the ciliary sulcus.

In an example implementation, using the scleral spur, iridocorneal angle, or other AI located anatomy as a fiduciary, the process flow 1300 may determine iris zonule distance (IZD), trabecular ciliary process distance (TCPD), trabecular iris area (TIA), and/or iris-lens angle (ILA). In some example aspects, in association with determining the iris zonule distance (IZD) or trabecular ciliary process distance (TCPD), acquiring image data at 1305 may be implemented using an imaging technique and/or imaging device capable of imaging through the iris.

At 1340, the process flow 1300 may include determining a presence, an absence, a progression, or a stage of a disease of the eye based on one or more located anatomy (as described with reference to 1310, 1320, and 1330) and/or one or more measurements (as described with reference to 1315, 1325, and 1335) described herein. In some other examples, determining the presence, the absence, the progression, or the stage of the disease may be based at least in part on a change in location of the anatomy and/or a change in the one or more measurements.

The process flow 1300 may include determining the presence, the absence, the progression, or the stage of the disease using one or more machine learning techniques (e.g., machine learning models, artificial intelligence, etc.) described herein. The output provided using the one or more machine learning techniques may be referred to as AI generated predictions of the presence, the absence, the progression, or the stage of the disease.

In an example of determining the stage of a disease, the systems and techniques described herein may support classifying patients having a certain stage of a disease (e.g., Stage 0 to Stage 4, with Stage 0 indicating healthy, and Stage 4 being the most severe stage of the disease). The systems and techniques may include providing the stage to a clinician in association with deriving a treatment strategy or providing treatment. In some aspects, the systems and techniques may support deriving the treatment strategy (e.g., providing treatment recommendations) based on the stage of the disease.

For example, aspects of the present disclosure described herein may include generating predictions (e.g., of the presence, the absence, the progression, or the stage of a disease) and probability scores and/or confidence scores associated with the predictions. In an example, the techniques described herein may include outputting a prediction (e.g., presence, absence, a progression, or a stage of a disease) in combination with a corresponding probability score and/or confidence score. In some aspects, the techniques described herein may include outputting the prediction for cases in which a corresponding probability score and/or confidence score associated with the prediction is equal to or greater than a threshold value. In some additional and/or alternative aspects, the techniques described herein may include outputting temporal information associated with the prediction (e.g., expected onset of a disease) in combination with a corresponding probability score and/or confidence score.

The terms “locating” and “detecting” may include determining location information of an object (e.g., a target structure, anatomy, etc.) described herein using, for example, object detection, computer vision, pixel masks, bounding boxes, and the like as described herein.

Referring to FIG. 14, at 1405-a, the process flow 1400 may include acquiring image data of an eye of a patient (e.g., from a database, data repository, PACS/DICOM type system, and the like as described herein). Additionally, or alternatively, at 1405-b, the process flow 1400 may include generating image data of an eye of a patient based on one or more imaging signals.

In some aspects, the image data includes one or more images generated based on one or more imaging signals, the one or more imaging signals including ultrasound pulses; and the image data includes a B-scan of the eye of the patient.

In some aspects, the image data includes one or more images generated based on one or more imaging signals, the one or more imaging signals including infrared laser light; and the image data includes a B-scan of the eye of the patient.

At 1410, the process flow 1400 may include locating one or more target structures included in an eye of a patient based on processing image data of the eye of the patient.

In some aspects, the one or more target structures include at least one of: tissue included in the eye; surgically modified tissue included in the eye; pharmacologically modified tissue included in the eye; and an implant included in the eye. In some examples, the one or more target structures may include at least one of: a cornea, a scleral wall, a scleral spur, an iris, a natural lens, a zonule, a ciliary body, a ciliary muscle, surgically modified tissue, and an implant.

In some aspects, processing the image data includes: providing (at 1415) at least a portion of the image data to one or more machine learning models; and receiving (at 1420) an output in response to the one or more machine learning models processing at least the portion of the image data, wherein the output includes location data of the one or more target structures. For example, the one or more machine learning models may detect the one or more target structures and provide the location data in response to detecting the one or more target structures.

In some aspects, processing the image data involves processing (e.g., converting) the image data into a format suitable for input into an artificial intelligence model.

In some aspects, the image data includes a set of pixels; and processing at least the portion of the image data by the one or more machine learning models includes: generating encoded image data in response to processing at least the portion of the image data using a set of encoder filters; and generating a mask image in response to processing at least the portion of the encoded image data using a set of decoder filters, wherein the mask image includes an indication of one or more pixels, included among the set of pixels included in the image data, that are associated with the one or more target structures.

In some aspects, the output from the one or more machine learning models includes one or more predicted masks; and determining the location data, the one or more measurements, or both is based on the one or more predicted masks.

At 1425, the process flow 1400 may include determining one or more measurements associated with an anterior portion of the eye, based on the location data and one or more characteristics associated with the one or more target structures.

In an example, the one or more measurements include at least one of: a measurement with respect to at least one axis of a set of axes associated with the eye; an angle between two or more axes of the set of axes; and a second measurement associated with an implant included in the eye.

In some aspects, the one or more measurements are associated with a first region posterior to an iris of the eye, a second region anterior to the iris, or both.

In some aspects, wherein the one or more measurements include at least one of: anterior chamber depth; iris thickness; iris-to-lens contact distance; iris zonule distance; trabecular ciliary process distance; and trabecular iris space area; and a measurement associated with an implant included in the eye.

In some examples, the one or more measurements include at least one of: corneal thickness; a meridian associated with observing the eye; an angle between a pupillary axis and a visual axis associated with the eye; at least one of an anterior radius and a posterior radius of a cornea of the eye; at least one of an anterior radius, a posterior radius, and a thickness of a natural lens of the eye; and a distance between a posterior cornea and anterior lens of the eye with respect to a visual axis associated with the eye.

At 1430, the process flow 1400 may include determining a presence, an absence, a progression, or a stage of a disease of the eye based on the one or more measurements. In some other examples, determining the presence, the absence, the progression, or the stage of the disease may be based at least in part on a change in the one or more measurements.

In an example, determining the presence, the absence, the progression, or the stage is based on a correlation between the one or more measurements and the disease.

In another example, determining the presence, the absence, the progression, or the stage is based on a probability of the disease of the eye. For example, at 1435, the process flow 1400 may include providing the one or more measurements to the one or more machine learning models. At 1440, the process flow 1400 may include receiving a second output in response to the one or more machine learning models processing the one or more measurements. In an example, the second output includes the probability of the disease of the eye.

In another example, the process flow 1400 includes determining a change in intraocular pressure in the eye based on the one or more measurements, wherein determining the presence, the absence, the progression, or the stage of the disease is based on the intraocular pressure.

Aspects of the process flow 1400 include training the one or more machine learning models based on a training data set. The training data set may include at least one of: reference image data associated with at least one eye of one or more reference patients; label data associated with the one or more target structures; one or more reference masks for classifying pixels included in the reference image data in association with locating the one or more target structures; and image classification data corresponding to at least one image of a set of reference images. In some aspects, the reference image data, the label data, the one or more reference masks, and the image classification data are associated with a pre-operative state, an intraoperative state, a post-operative state, a disease state, or a combination thereof.

Any of the steps, functions, and operations discussed herein can be performed continuously and automatically.

The exemplary systems and methods of this disclosure have been described in relation to examples of a system 1100, a device 1105, an imaging device 1107, and a server 1110. However, to avoid unnecessarily obscuring the present disclosure, the preceding description omits a number of known structures and devices. This omission is not to be construed as a limitation of the scope of the claimed disclosure. Specific details are set forth to provide an understanding of the present disclosure. It should, however, be appreciated that the present disclosure may be practiced in a variety of ways beyond the specific detail set forth herein.

Furthermore, while the exemplary embodiments illustrated herein show the various components of the system collocated, certain components of the system can be located remotely, at distant portions of a distributed network, such as a LAN and/or the Internet, or within a dedicated system. Thus, it should be appreciated, that the components of the system can be combined into one or more devices, such as a server, communication device, or collocated on a particular node of a distributed network, such as an analog and/or digital telecommunications network, a packet-switched network, or a circuit-switched network. It will be appreciated from the preceding description, and for reasons of computational efficiency, that the components of the system can be arranged at any location within a distributed network of components without affecting the operation of the system.

Furthermore, it should be appreciated that the various links connecting the elements can be wired or wireless links, or any combination thereof, or any other known or later developed element(s) that is capable of supplying and/or communicating data to and from the connected elements. These wired or wireless links can also be secure links and may be capable of communicating encrypted information. Transmission media used as links, for example, can be any suitable carrier for electrical signals, including coaxial cables, copper wire, and fiber optics, and may take the form of acoustic or light waves, such as those generated during radio-wave and infra-red data communications.

While the flowcharts have been discussed and illustrated in relation to a particular sequence of events, it should be appreciated that changes, additions, and omissions to this sequence can occur without materially affecting the operation of the disclosed embodiments, configuration, and aspects.

A number of variations and modifications of the disclosure can be used. It would be possible to provide for some features of the disclosure without providing others.

In yet another embodiment, the systems and methods of this disclosure can be implemented in conjunction with a special purpose computer, a programmed microprocessor or microcontroller and peripheral integrated circuit element(s), an ASIC or other integrated circuit, a digital signal processor, a hard-wired electronic or logic circuit such as discrete element circuit, a programmable logic device or gate array such as PLD, PLA, FPGA, PAL, special purpose computer, any comparable means, or the like. In general, any device(s) or means capable of implementing the methodology illustrated herein can be used to implement the various aspects of this disclosure. Exemplary hardware that can be used for the present disclosure includes computers, handheld devices, telephones (e.g., cellular, Internet enabled, digital, analog, hybrids, and others), and other hardware known in the art. Some of these devices include processors (e.g., a single or multiple microprocessors), memory, nonvolatile storage, input devices, and output devices. Furthermore, alternative software implementations including, but not limited to, distributed processing or component/object distributed processing, parallel processing, or virtual machine processing can also be constructed to implement the methods described herein.

In yet another embodiment, the disclosed methods may be readily implemented in conjunction with software using object or object-oriented software development environments that provide portable source code that can be used on a variety of computer or workstation platforms. AI ternatively, the disclosed system may be implemented partially or fully in hardware using standard logic circuits or VLSI design. Whether software or hardware is used to implement the systems in accordance with this disclosure is dependent on the speed and/or efficiency requirements of the system, the particular function, and the particular software or hardware systems or microprocessor or microcomputer systems being utilized.

In yet another embodiment, the disclosed methods may be partially implemented in software that can be stored on a non-transitory computer readable storage medium, executed on programmed general-purpose computer with the cooperation of a controller and memory, a special purpose computer, a microprocessor, or the like. In these instances, the systems and methods of this disclosure can be implemented as a program embedded on a personal computer such as an applet, JAVA® or CGI script, as a resource residing on a server or computer workstation, as a routine embedded in a dedicated measurement system, system component, or the like. The system can also be implemented by physically incorporating the system and/or method into a software and/or hardware system.

AI though the present disclosure describes components and functions implemented in the embodiments with reference to particular standards and protocols, the disclosure is not limited to such standards and protocols. Other similar standards and protocols not mentioned herein are in existence and are considered to be included in the present disclosure. Moreover, the standards and protocols mentioned herein and other similar standards and protocols not mentioned herein are periodically superseded by faster or more effective equivalents having essentially the same functions. Such replacement standards and protocols having the same functions are considered equivalents included in the present disclosure.

The present disclosure, in various embodiments, configurations, and aspects, includes components, methods, processes, systems and/or apparatus substantially as depicted and described herein, including various embodiments, subcombinations, and subsets thereof. Those of skill in the art will understand how to make and use the systems and methods disclosed herein after understanding the present disclosure. The present disclosure, in various embodiments, configurations, and aspects, includes providing devices and processes in the absence of items not depicted and/or described herein or in various embodiments, configurations, or aspects hereof, including in the absence of such items as may have been used in previous devices or processes, e.g., for improving performance, achieving ease, and/or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments, configurations, or aspects for the purpose of streamlining the disclosure. The features of the embodiments, configurations, or aspects of the disclosure may be combined in alternate embodiments, configurations, or aspects other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment, configuration, or aspect. Thus, the following claims are hereby incorporated into this Detailed Description, with each claim standing on its own as a separate preferred embodiment of the disclosure.

Moreover, though the description of the disclosure has included description of one or more embodiments, configurations, or aspects and certain variations and modifications, other variations, combinations, and modifications are within the scope of the disclosure, e.g., as may be within the skill and knowledge of those in the art, after understanding the present disclosure. It is intended to obtain rights, which include alternative embodiments, configurations, or aspects to the extent permitted, including alternate, interchangeable and/or equivalent structures, functions, ranges, or steps to those claimed, whether or not such alternate, interchangeable and/or equivalent structures, functions, ranges, or steps are disclosed herein, and without intending to publicly dedicate any patentable subject matter.

The phrases “at least one,” “one or more,” “or,” and “and/of” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The term “automatic” and variations thereof, as used herein, refers to any process or operation, which is typically continuous or semi-continuous, done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

Aspects of the present disclosure may take the form of an embodiment that is entirely hardware, an embodiment that is entirely software (including firmware, resident software, micro-code, etc.) or an embodiment combining software and hardware aspects that may all generally be referred to herein as a “circuit,” “module,” or “system.” Any combination of one or more computer-readable medium(s) may be utilized. The computer-readable medium may be a computer-readable signal medium or a computer-readable storage medium.

A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, or any suitable combination thereof. A computer-readable signal medium may be any computer-readable medium that is not a computer-readable storage medium and that can communicate, propagate, or transport a program for use by or in connection with an instruction execution system, apparatus, or device. Program code embodied on a computer-readable medium may be transmitted using any appropriate medium, including, but not limited to, wireless, wireline, optical fiber cable, RF, etc., or any suitable combination of the foregoing.

The terms “determine,” “calculate,” “compute,” and variations thereof, as used herein, are used interchangeably and include any type of methodology, process, mathematical operation or technique.

What is claimed is:

1. A method comprising:
locating one or more target structures comprised in an eye of a patient based on processing image data of the eye of the patient, wherein processing the image data comprises:
providing at least a portion of the image data to one or more machine learning models; and
receiving an output from the one or more machine learning models in response to the one or more machine learning models processing at least the portion of the image data, wherein the output comprises location data of the one or more target structures;
determining one or more measurements associated with an anterior portion of the eye, based on the location data and one or more characteristics associated with the one or more target structures; and
determining a presence, an absence, a progression, or a stage of a disease of the eye based on the one or more measurements.

2. The method of claim 1, wherein determining the presence, the absence, the progression, or the stage is based on a correlation between the one or more measurements and the disease.

3. The method of claim 1, further comprising:
providing the one or more measurements to the one or more machine learning models; and
receiving a second output in response to the one or more machine learning models processing the one or more measurements,
wherein:
the second output comprises a probability of the disease of the eye; and
determining the presence, the absence, the progression, or the stage is based on the probability.

4. The method of claim 1, wherein:
the output from the one or more machine learning models comprises one or more predicted masks; and
determining the location data, the one or more measurements, or both is based at least in part on the one or more predicted masks.

5. The method of claim 1, wherein the one or more measurements comprise at least one of:
a measurement with respect to at least one axis of a set of axes associated with the eye;
an angle between two or more axes of the set of axes; and
a second measurement associated with an implant comprised in the eye.

6. The method of claim 1, wherein the one or more target structures comprise at least one of:
tissue comprised in the eye;
surgically modified tissue comprised in the eye;
pharmacologically modified tissue comprised in the eye; and
an implant comprised in the eye.

7. The method of claim 1, further comprising:
determining a change in intraocular pressure in the eye based on the one or more measurements, wherein determining the presence, the absence, the progression, or the stage of the disease is based on the intraocular pressure.

8. The method of claim 1, wherein:
the one or more measurements are associated with a first region posterior to an iris of the eye, a second region anterior to the iris, or both.

9. The method of claim 1, wherein:
the image data comprises one or more images generated based on one or more imaging signals, the one or more imaging signals comprising ultrasound pulses; and
the image data comprises a B-scan of the eye of the patient.

10. The method of claim 1, wherein:
the image data comprises one or more images generated based on one or more imaging signals, the one or more imaging signals comprising infrared laser light; and
the image data comprises a B-scan of the eye of the patient.

11. The method of claim 1, wherein the one or more measurements comprise at least one of:
anterior chamber depth;
iris thickness;
iris-to-lens contact distance;
iris zonule distance;
trabecular ciliary process distance; and
trabecular iris space area; and
a measurement associated with an implant comprised in the eye.

12. The method of claim 1, further comprising training the one or more machine learning models based on a training data set, the training data set comprising at least one of:
reference image data associated with at least one eye of one or more reference patients;
label data associated with the one or more target structures;
one or more reference masks for classifying pixels included in the reference image data in association with locating the one or more target structures; and
image classification data corresponding to at least one image of a set of reference images,
wherein the reference image data, the label data, the one or more reference masks, and the image classification data are associated with a pre-operative state, an intraoperative state, a post-operative state, a disease state, or a combination thereof.

13. The method of claim 1, wherein:
the image data comprises a set of pixels; and
processing at least the portion of the image data by the one or more machine learning models comprises:
generating encoded image data in response to processing at least the portion of the image data using a set of encoder filters; and
generating a mask image in response to processing at least the portion of the encoded image data using a set of decoder filters,
wherein the mask image comprises an indication of one or more pixels, included among the set of pixels comprised in the image data, that are associated with the one or more target structures.

14. An apparatus comprising:
a processor; and
memory in electronic communication with the processor, wherein instructions stored in the memory are executable by the processor to:
locate one or more target structures comprised in an eye of a patient based on processing image data of the eye of the patient, wherein processing the image data comprises:
providing at least a portion of the image data to one or more machine learning models; and
receiving an output from the one or more machine learning models in response to the one or more machine learning models processing at least the portion of the image data, wherein the output comprises location data of the one or more target structures;
determine one or more measurements associated with an anterior portion of the eye, based on the location data and one or more characteristics associated with the one or more target structures; and
determine a presence, an absence, a progression, or a stage of a disease of the eye based on the one or more measurements.

15. The apparatus of claim 14, wherein determining the presence, the absence, the progression, or the stage is based on a correlation between the one or more measurements and the disease.

16. The apparatus of claim 14, wherein the instructions are further executable by the processor to:
provide the one or more measurements to the one or more machine learning models; and
receive a second output in response to the one or more machine learning models processing the one or more measurements,
wherein:
the second output comprises a probability of the disease of the eye; and
determining the presence, the absence, the progression, or the stage is based on the probability

17. The apparatus of claim 14, wherein:
the output from the one or more machine learning models comprises one or more predicted masks; and
determining the location data, the one or more measurements, or both is based at least in part on the one or more predicted masks.

18. The apparatus of claim 14, wherein the one or more measurements comprise at least one of:
a measurement with respect to at least one axis of a set of axes associated with the eye;
an angle between two or more axes of the set of axes; and
a second measurement associated with an implant comprised in the eye.

19. The apparatus of claim 14, wherein the one or more target structures comprise at least one of:
tissue comprised in the eye;
surgically modified tissue comprised in the eye;
pharmacologically modified tissue comprised in the eye; and
an implant comprised in the eye.

20. A non-transitory computer readable medium comprising instructions, which when executed by a processor:
generates image data of an eye of a patient based on one or more imaging signals;
locates one or more target structures comprised in an eye of a patient based on processing image data of the eye of the patient, wherein processing the image data comprises:
providing at least a portion of the image data to one or more machine learning models; and
receiving an output from the one or more machine learning models in response to the one or more machine learning models processing at least the portion of the image data, wherein the output comprises location data of the one or more target structures;
determines one or more measurements associated with an anterior portion of the eye, based on the location data and one or more characteristics associated with the one or more target structures; and
determines a presence, an absence, a progression, or a stage of a disease of the eye based on the one or more measurements.
